(19) United States
(12) Patent Application Publication
(10) Pub. No.: US 2026/0345218 A1
(43) Pub. Date: Nov. 5, 2026

(54) CRYOTHERAPEUTIC DEVICES, SYSTEMS, AND METHODS FOR TARGETED LUNG NEUROMODULATION

(71) Applicant: Ryme Medical, Inc., Mountain View, CA (US)

(72) Inventors: Katherine Miyashiro, San Francisco, CA (US); Emily Schoenhoff, Sunnyvale, CA (US); Nrupen Zaver, Sunnyvale, CA (US); Austin Haynes, Mountain View, CA (US); Jesus Magaña, Redwood City, CA (US); Paul Andreotti, Santa Rosa, CA (US); Bernard Andreas, Los Altos, CA (US); Erin McDowell, Rancho Mission Viejo, CA (US)

(21) Appl. No.: 19/557,018

(22) Filed: Mar. 4, 2026

Related U.S. Application Data
(60) Provisional application No. 63/766,862, filed on Mar. 4, 2025.

(51) Int. Cl. A61B 18/02; A61B 1/00; A61B 1/018; A61B 1/267; A61B 17/00; A61B 18/00; A61B 90/00

(52) U.S. Cl. CPC A61B 18/02; A61B 1/00128; A61B 1/018; A61B 1/2676; A61B 90/06; A61B 90/39; A61B 2017/00539; A61B 2017/00557; A61B 2018/00041; A61B 2018/00101; A61B 2018/00172; A61B 2018/00184; A61B 2018/00434; A61B 2018/00541; A61B 2018/00577; A61B 2018/00714; A61B 2018/00744; A61B 2018/00791; A61B 2018/00982; A61B 2018/0212; A61B 2018/0231; A61B 2018/0262; A61B 2090/064; A61B 2090/3966

(57) ABSTRACT

Cryotherapeutic devices, systems, and methods for targeted lung neuromodulation are disclosed herein. A cryotherapeutic device configured in accordance with embodiments of the present technology can include, for example, an elongated catheter shaft configured to deliver a cooling assembly at a distal portion of the elongated catheter shaft to a target site of within the bronchus. The cooling assembly can include an expandable member configured to receive a refrigerant that, upon expansion within the expandable member, imparts cryogenic cooling to bronchial tissue at the target site. This cryogenic cooling is expected to ablate neural fibers proximate to the target site to treat various bronchial conditions, including COPD. The device can be sized and shaped to extend through a working channel of a bronchoscope such that at least a portion of the cooling assembly can be visualized during a cryotherapeutic procedure.

### CROSS-REFERENCE TO RELATED APPLICATION

This application claims the benefit of U.S. Provisional Patent Application No. 63/766,862, titled “CRYOTHERAPEUTIC DEVICES, SYSTEMS, AND METHODS FOR TARGETED LUNG NEUROMODULATION,” and filed Mar. 4, 2025, which is incorporated herein by reference in its entirety.

### TECHNICAL FIELD

The present technology relates generally to targeted lung denervation. Several embodiments are directed to cryotherapeutic devices, systems, and methods for targeted lung neuromodulation.

### BACKGROUND

Pulmonary diseases are characterized as pathologies that adversely affect performance of the lungs, making it difficult for air-breathing animals (e.g., humans) to perform the gas exchange necessary for survival. Certain pulmonary diseases, such as chronic obstructive pulmonary disease (“COPD”), asthma, and bronchiectasis, specifically affect the airways that carry oxygen into the body. For example, COPD and asthma are characterized by a narrowing or blockage of airways which can be the result of the thickening of airway walls, alterations in the structures within or around the airway walls, or combinations thereof. For example, blockages of airway lumens can be caused by excessive intraluminal mucus and/or edema fluid. Airway wall thickening can result from inflammation, contraction or hypertrophy of the airway smooth muscle, mucous gland hypertrophy, and/or edema. Structural changes around the airway can reduce radial traction on the airway wall and lead to a narrowing of the airway. This narrowing or obstruction of airways can significantly decrease the amount of gas exchanged in the lungs resulting in breathlessness.

COPD is characterized by airflow limitation and long-term respiratory symptoms, such as shortness of breath and cough. The two most common types of COPD are emphysema and chronic bronchitis. Emphysema is defined as enlarged airspaces (alveoli) with broken down walls resulting in permanent damage to the lung tissue. Chronic bronchitis is defined as a productive cough (i.e., producing mucus or phlegm) for an extended period of time.

### BRIEF DESCRIPTION OF THE DRAWINGS

FIG. 1A is a partially schematic illustration of a cryotherapy system configured in accordance with embodiments of the technology.

FIG. 1B is an enlarged side view of a cryotherapeutic device of the cryotherapeutic system of FIG. 1A.

FIG. 1C is an enlarged, partial cutaway view of a cooling assembly of the cryotherapeutic device of FIG. 1B.

FIG. 1D is a cross-sectional view illustrating the flow path of refrigerant along the cryotherapeutic device of FIG. 1B in accordance with embodiments of the present technology.

FIG. 1E is an enlarged view of the distal portion of the cryotherapeutic device shown in FIG. 1D.

FIG. 1F is a perspective view a handle of the therapeutic device of FIG. 1B coupled to an endoscope in accordance with embodiments of the present technology.

FIG. 2 is an enlarged view of a portion of an injection tube for a cryotherapeutic device configured in accordance with embodiments of the present technology.

FIG. 3A illustrates treatment locations for cryotherapeutic procedures in accordance with some embodiments of the technology.

FIG. 3B illustrates a distal portion of a cryotherapeutic device at a treatment location in an airway in accordance with embodiments of the present technology.

FIG. 4 illustrates end-view images of a cooling assembly of a cryotherapeutic device in the airway during various stages of a cryotherapeutic procedure in accordance with some embodiments of the technology.

FIG. 5 is a block diagram of a processing device configured to perform control of a cryotherapeutic system in accordance with some embodiments of the technology.

FIG. 6A is a cross-sectional side view of an injection tube for a cryotherapeutic device configured in accordance with embodiments of the present technology.

FIG. 6B is a side view of an injection tube for a cryotherapeutic device configured in accordance with embodiments of the present technology.

FIG. 6C is a cross-sectional side view of an injection tube for a cryotherapeutic device configured in accordance with embodiments of the present technology.

FIG. 6D is a side view of an injection tube within an expandable member at a distal end portion of a cryotherapeutic device in accordance with embodiments of the present technology.

FIG. 6E illustrates a cross-sectional side view of an injection tube assembly configured in accordance with embodiments of the present technology.

FIG. 6F is a side view of an injection tube and an expandable member of a cryotherapeutic device in accordance with embodiments of the present technology.

FIG. 6G illustrates schematic side views of injection tubes for cryotherapeutic devices configured in accordance with embodiments of the present technology.

FIG. 6H illustrates partially transparent isometric and side views of a therapeutically cooled region along a body lumen treated using a cryotherapeutic system configured in accordance with embodiments of the present technology.

FIG. 6I is a partially cross-sectional side view of a distal portion of a cryotherapeutic device configured in accordance with embodiments of the present technology.

FIG. 7A is a partially cross-sectional side view of a distal portion of a cryotherapeutic device configured in accordance with some embodiments of the technology.

FIG. 7B is a partially cross-sectional side view of a distal portion of a cryotherapeutic device configured in accordance with some embodiments of the technology.

FIG. 7C illustrates schematic front view and side view of a distal portion of a cryotherapeutic device configured in accordance with some embodiments of the technology.

FIG. 8A is a cross-sectional end view of an expandable member configured in accordance with some embodiments of the technology.

FIG. 8B a side view of a corresponding injection tube in accordance with some embodiments of the technology.

FIG. 9A is cross-sectional end-on view of a non-occlusive expandable member configured in accordance with some embodiments of the technology.

FIG. 9B is a cross-sectional end-on view of the expandable member positioned in an airway lumen configured in accordance with some embodiments of the technology.

FIG. 10 illustrates an end-on cross-sectional view of an expandable member configured in accordance with some embodiments of the technology.

FIG. 11A is a side view of a balloon for cryotherapeutic devices configured in accordance with some embodiments of the technology.

FIG. 11B illustrates a cross-sectional view of a balloon for cryotherapeutic devices configured in accordance with some embodiments of the technology.

FIG. 11C is a side view of a balloon for cryotherapeutic devices configured in accordance with some embodiments of the technology.

FIG. 11D is a side view of a balloon for cryotherapeutic devices configured in accordance with some embodiments of the technology.

FIG. 12A is a partially cross-sectional side view of a distal portion of a cryotherapeutic device configured in accordance with some embodiments of the technology.

FIG. 12B is a cross-sectional side view of a distal portion of a cryotherapeutic device configured in accordance with some embodiments of the technology.

FIG. 12C is a cross-sectional end-on view of a distal portion of a cryotherapeutic device configured in accordance with some embodiments of the technology.

FIG. 13 is a side view of a distal portion of a cryotherapeutic device configured in accordance with some embodiments of the technology.

FIGS. 14A and 14B are side views of distal portions of cryotherapeutic devices having dual balloon structures in accordance with some embodiments of the technology.

FIG. 15A is a partially schematic side view of a distal portion of a cryotherapeutic device configured to provide narrow thermal profiles in accordance with some embodiments of the technology.

FIG. 15B is a side view of a distal portion of a cryotherapeutic device configured to provide narrow thermal profiles in accordance with some embodiments of the technology.

FIG. 15C is a partially schematic side view of an injection tube for a cryotherapeutic device configured to provide narrow thermal profiles in accordance with some embodiments of the technology.

FIG. 15D illustrates a side view of a narrow thermal profile achieved by an expandable member in accordance with some embodiments of the technology.

FIG. 16A is a side view of a distal portion of a cryotherapeutic device in accordance with some embodiments of the technology.

FIG. 16B illustrates partially schematic side views of distal portions of cryotherapeutic devices in accordance with some embodiments of the technology.

FIG. 17A illustrates a distal end portion of a cryotherapeutic device including a balloon around an injection tube configured in accordance with the present technology.

FIG. 17B illustrates a distal end portion of a cryotherapeutic device including a balloon around an injection tube configured in accordance with the present technology.

FIG. 17C is a side view of a distal end portion of a cryotherapeutic device including a balloon around an injection tube configured in accordance with the present technology.

FIG. 17D illustrates schematic views of a distal portion of a cryotherapeutic device including a balloon around an injection tube configured in accordance with the present technology.

FIG. 17E illustrates schematic views of a distal portion of a cryotherapeutic device including a balloon around an injection tube configured in accordance with the present technology.

FIG. 18A illustrates a side view and a perspective view of a distal portion of a cryotherapeutic device configured in accordance with embodiments of the technology.

FIG. 18B is an enlarged view of an injection tube configuration of the device of FIG. 18A.

FIG. 18C illustrates a partially schematic side view of a distal portion of a cryotherapeutic device configured in accordance with embodiments of the technology.

FIG. 18D illustrates a partially schematic side view of a distal portion of a cryotherapeutic device configured in accordance with embodiments of the technology.

FIG. 19A illustrates a cross-sectional view of a cryotherapeutic device including an exhaust shaft and an injection tube located therein configured in accordance with embodiments of the technology.

FIG. 19B illustrates a cross-sectional view of a cryotherapeutic device including an exhaust shaft and an injection tube located therein configured in accordance with embodiments of the technology.

FIG. 20A illustrates a schematic side view of a cryotherapeutic device including an exhaust shaft and an injection tube configured in accordance with embodiments of the technology.

FIG. 20B illustrates a cross-sectional view of a cryotherapeutic device including an exhaust shaft and an injection tube configured in accordance with embodiments of the technology.

### DETAILED DESCRIPTION

Cryotherapeutic systems and methods for targeted lung neuromodulation therapies are disclosed herein. In some embodiments, for example, cryotherapeutic systems can include a cooling assembly (also referred to as an “end effector”) configured to be positioned at target sites within a patient's airways and provide cryotherapeutic cooling to modulate nerves (e.g., ablate, dennervate) at the target sites for the treatment of COPD and/or other pulmonary diseases. The cooling assembly can include an expandable member (e.g., a balloon) and a refrigerant delivery tube (also referred to as an injection tube, delivery lumen, and the like) with one or more outlet ports that are fluidically coupled to a liquid refrigerant source. Upon exiting the outlet port, the liquid refrigerant can rapidly expand within the expandable member in a manner suitable for delivering cryotherapeutic cooling to nerves at or proximate the target site. In some embodiments, the cryotherapeutic device may be configured for use with a bronchoscope in a cryotherapy procedure such that a user (e.g., a physician performing the procedure) may visualize the target site before, during, and/or after the procedure.

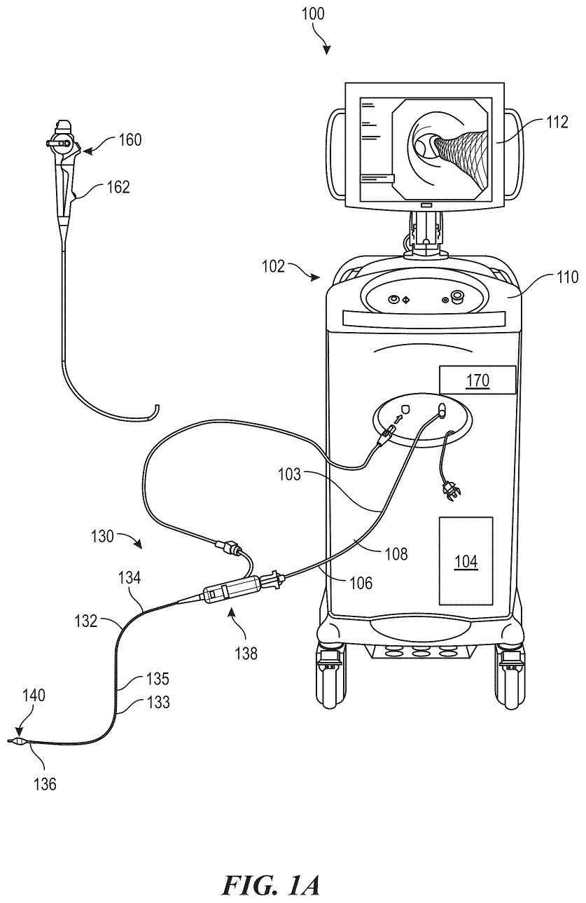

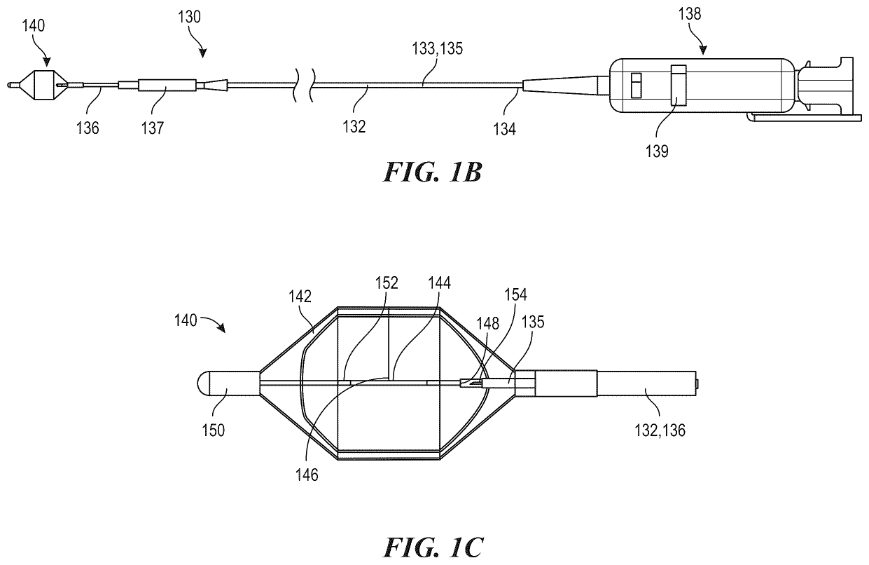

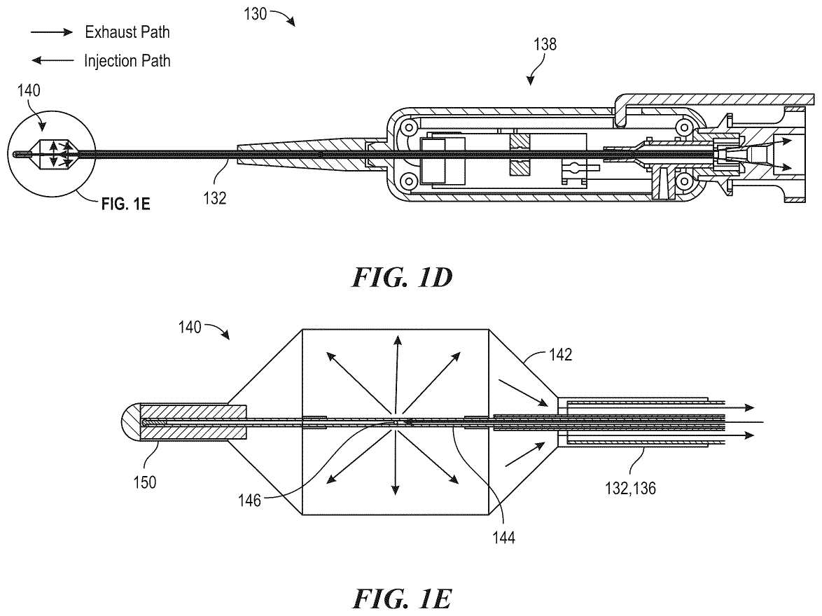

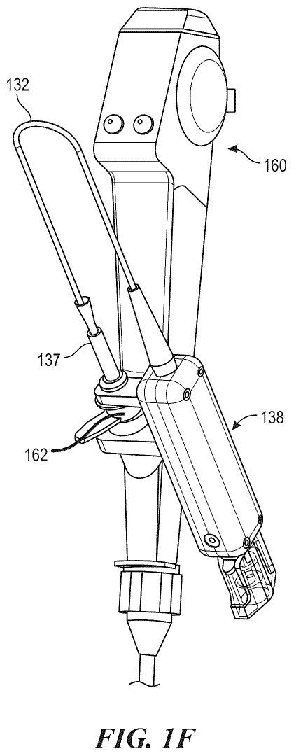

Specific details of several embodiments of the technology are described below with reference to FIGS. 1A-20B. Although many of the embodiments are described below with respect to devices, systems, and methods for treatment and monitoring during cryotherapy for the treatment of COPD, other applications, and other embodiments in addition to those described herein are within the scope of the technology. For example, the present technology may be used for the treatment of different indications (e.g., asthma) and/or different target sites within a patient's body other than the airways, such as other portions of the respiratory system and/or other portions of the body. Additionally, several other embodiments of the technology can have different configurations, components, or procedures than those described herein, and that features of the embodiments shown can be combined with one another. A person of ordinary skill in the art, therefore, will accordingly understand that the technology can have other embodiments with additional elements, or the technology can have other embodiments without several of the features shown and described below with reference to the Figures.

With regard to the terms “distal” and “proximal” within this description, unless otherwise specified, the terms can reference a relative position of the portions of a cryotherapeutic device and/or an associated delivery device with reference to an operator and/or a location in the bronchus. For example, proximal can refer to a position closer to the operator of the device or an associated access point (e.g., the mouth), and distal can refer to a position that is more distant from the operator of the device or further from the access point.

### Bronchial Neuromodulation

Bronchial neuromodulation is the partial or complete incapacitation or other effective disruption of nerves innervating portions of the bronchi. In particular, bronchial neuromodulation includes inhibiting, reducing, and/or blocking neural communication along neural fibers (i.e., efferent and/or afferent nerve fibers) innervating the select bronchi. Such incapacitation can be long-term (e.g., permanent or for periods of months, years, or decades) or short-term (e.g., for periods of minutes, hours, days, or weeks). Bronchial neuromodulation is expected to efficaciously treat several clinical conditions characterized by increased activity of airway smooth muscle and mucus producing glands within the airways, such as COPD. The neuromodulation is expected to relax the airway smooth muscle, decrease mucus production, and/or decrease nervous system mediated inflammation and edema, which results in the reduction of airway obstruction and makes it easier to breathe.

As disclosed herein, cryotherapy can be used to partially or completely incapacitate neural pathways, such as those innervating the bronchi. Cryotherapy includes cooling tissue at a target site in a manner that modulates neural function. The mechanisms of cryotherapeutic tissue damage include, for example, direct cell injury (e.g., necrosis) and sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Several embodiments of the present technology include cooling a structure at or near an inner surface of a wall of an airway such that proximate (e.g., adjacent) tissue is effectively cooled to a depth where nerves innervating the lungs reside (e.g., the pulmonary plexus). For example, the cooling structure is cooled to the extent that it causes therapeutically effective, cryogenic neural modulation. Sufficiently cooling at least a portion of a nerve is expected to slow or potentially block conduction of neural signals to produce a prolonged or permanent reduction in neural activity.

Cryotherapy has certain characteristics that can be beneficial for intra airway neuromodulation. For example, cryotherapy typically does not cause significant collagen tightening, and thus cryotherapy is not typically associated with chronic airway stenosis. Further, cryotherapy systems and methods disclosed herein allow the airways to maintain their cellular architecture while debilitating the cellular function and causing targeted cell death, thereby maintaining the mechanical integrity of the affected tissues.

Cryotherapies generally operate at temperatures that cause cryotherapeutic applicators to adhere to tissue. This can be beneficial because it promotes stable, consistent, and continued contact with the tissue along the inner wall of the bronchi during treatment. The typical conditions of treatment can make this an attractive feature because, for example, respiration causes the bronchi to rise and fall and thereby moves the target site, a patient can move during treatment, and/or a catheter associated with the cryotherapeutic applicator can move. Further, in procedures where a patient is asked to hold his/her breath to keep the target site stationary, the patient can exhale once the applicator is adhered to the target site. This reduces the duration of breath holding, which can often be difficult for those patients suffering from COPD or other pulmonary diseases the procedure is used to treat. Adhesion associated with cryotherapeutic cooling also can be advantageous when treating short bronchi in which stable intraluminal positioning can be more difficult to achieve.

Further, existing treatments of a pulmonary disorder include use of systemic or inhaled medication, such as corticosteroids, antibiotics, bronchodilators, aerosol delivery of a “mucolytic” agent (e.g., water, hypertonic saline solution). These medications often require prolonged use. Thermal ablation treatments, such as bronchial thermoplasty and/or other RF ablation treatments, involve destroying smooth muscle tone by ablating (e.g., via heat) the airway wall in bronchial branches within a lung, thereby eliminating both smooth muscles and nerves in the airway walls of the lung. The treated airways may be unable to respond to inhaled irritants, systemic hormones, and both local and central nervous system input. In contrast, cryotherapeutic systems disclosed herein have been shown to cryogenically ablate neural fibers for the treatment of COPD and/or other respiratory diseases, while preserving smooth muscle tone. It is also expected that the cryotherapeutic systems and methods disclosed herein may reduce the need for medications and/or enhance the efficacy of a patient's existing medications.

### Select Embodiments of Cryotherapy Systems and Methods

FIG. 1A is a partially schematic illustration of a cryotherapy system 100 (also referred to as a “cryotherapeutic system 100” or “system 100”) configured in accordance with embodiments of the technology; FIG. 1B is an enlarged side view of a cryotherapeutic device 130 of the system 100 of FIG. 1A, FIG. 1C is an enlarged, partial cutaway view of the cryotherapeutic device 130 of FIG. 1B, FIGS. 1D and 1E are cross-sectional view illustrating refrigerant flow paths along the cryotherapeutic device 130, and FIG. 1F is a perspective view of a proximal portion of the cryotherapeutic device 130.

Referring to FIG. 1A, the system 100 can include a console 102 operably coupled to the cryotherapeutic device 130. The console 102 can include a supply component 104 (shown schematically; also referred to as a “refrigerant source”) that retains a refrigerant, such as liquid nitrogen, nitrous oxide (N₂O), carbon dioxide, and/or a hydrofluorocarbon (“HFC”; e.g., Freon) at a desired pressure. The supply component 104 can be in fluid communication with the cryotherapeutic device 130 via a supply line 106 (e.g., a lumen, tube) to transport the refrigerant to the cryotherapeutic device 130. An exhaust line 108 (e.g., a lumen, tube) can also be coupled to the cryotherapeutic device 130 to receive the evaporated refrigerant (e.g., nitrogen gas) after the refrigerant expands during a cryotherapeutic procedure. In some embodiments, the exhaust line 108 is coupled to a pump (not shown; e.g., a vacuum pump) and/or a control valve at the console 102 to draw the evaporated refrigerant from the cryotherapeutic device 130. This reduces the backpressure of evaporated refrigerant and, in conjunction with the supply flow rate, can increase the refrigeration power.

Referring to FIGS. 1A and 1B together, the cryotherapeutic device 130 includes a shaft 132 having a proximal portion 134 and a distal portion 136, a handle 138 (also referred to as a “handle housing” or “housing”) at the proximal portion 134, and a cooling assembly 140 (also referred to as an “end effector” or “cryotherapeutic assembly”) at the distal portion 136 of the shaft 132. The supply line 106 can be in fluid communication with a delivery lumen 133 extending through the shaft 132 to deliver refrigerant to the cooling assembly 140, and the exhaust line 108 can be in fluid communication with an exhaust lumen 135 extending through or alongside the shaft 132 to receive refrigerant from the cooling assembly 140. In some embodiments, the supply line 106 and the exhaust line 108 define the delivery lumen 133 and exhaust lumen 135. The shaft 132 is configured to locate the distal portion 136 at a treatment site in the airways proximate (e.g., in or near) a desired site within the bronchus, and the cooling assembly 140 is configured to deliver a therapeutically effective cryogenic nerve modulation to the target site. The shaft 132 can apply cryogenic cooling (e.g., at temperatures of −15 °C to −80 °C, lower than −80 °C, or other suitable temperatures) for therapeutically effective neuromodulation at one or more target sites within the bronchus and is expected to relieve the symptoms of and/or treat various respiratory diseases, such as COPD. For example, embodiments of the cryotherapeutic devices disclosed herein can reach −80 °C, which has been shown to effectively target and therapeutically modulate nerves outside of the bronchial wall. Neural fibers within the bronchial walls can be targeted with higher temperatures.

As shown in FIGS. 1A and 1F, the cryotherapeutic device 130 is configured to be used with a bronchoscope 160 that can visualize the target site within the airways and aid in delivery of the cooling assembly 140 to the target site within the bronchus. For example, the distal portion of the bronchoscope 160 can be inserted into the nose or mouth of a subject (e.g., a human patient) to a position near a target site within the bronchus, and the distal portion 136 of the shaft 132 can be threaded through a working channel 162 of the bronchoscope 160 to navigate the cooling assembly 140 to a position at or near the target site. The cooling assembly 140 can then be exposed from the bronchoscope 160 (e.g., by moving the shaft 132 distal relative to a distal opening of the working channel 162 and/or retracting the bronchoscope 160), positioned to contact tissue at the target site, and deliver cryotherapeutic cooling to tissue at the target site. In such embodiments, the bronchoscope 160 can provide direct visualization to guide the cooling assembly 140 to one or more desired locations within the airways, as well as provide visualization during and/or after cryotherapeutic cooling. Suitable bronchoscopes 160 can include, but are not limited to, those manufactured by Olympus Corporation of Tokyo, Japan.

The supply component 104 can include a refrigerant cartridge for storing a refrigerant under pressure and/or a larger container (e.g., a tank, refillable cylinder, or the like) that retains a sufficient volume of refrigerant to perform multiple cryotherapy procedures. The refrigerant housed within the supply component 104 may be liquid nitrogen, carbon dioxide, a hydrofluorocarbon (“HFC”; e.g., Freon®, R-410A, etc.), and/or other suitable compressed or condensed refrigerants that can be retained in the supply component 104 at a sufficiently high pressure to maintain the refrigerant in at least a substantially liquid state at ambient temperatures. The supply component 104 is configured to retain the refrigerant at a desired pressure. For example, in one embodiment, liquid N₂O is contained in the supply container 104 at a pressure of 750 psi or greater so it is in at least a substantially liquid state at ambient temperatures. For example, when the refrigerant is liquid N2O, the supply component 104 can retain the refrigerant at or above a pressure threshold (e.g., 750 psi (5,171.07 kPa) or greater) to maintain the refrigerant in at least a substantially liquid state at ambient temperatures. In some embodiments, the supply component 104 can include one or more chambers (e.g., tanks, vessels) that receive the refrigerant exhaust (e.g., nitrogen gas) produced during cryotherapy. The supply component 104 can include features, such as one or more pumps or valves, configured to control the flow of the refrigerant from the supply component 104 to cooling assembly 140 at the distal portion 136 of the cryotherapeutic device 130. One or more flow sensors may be included in or coupled to the supply component 104, the supply line 106, the exhaust line 108, and/or the cooling assembly 140 for sensing or measuring refrigerant flow to and/or from the cryotherapeutic device 130. As shown in FIG. 1A, the supply component 104 is housed within or otherwise carried by the console 102 and coupled to the shaft 132 of the cryotherapeutic device 130 via an umbilical 103. In some embodiments, the supply component 104 can be separate from the console 102.

As further shown in FIG. 1A, the system 100 can further include a control component 110, such as a computing device, that receives information, data, signals, and/or other feedback from various components of the system 100 and, in some embodiments, provides the detected information to an operator before, during, and/or after a cryotherapeutic procedure. For example, the control component 110 can include a display 112 that provides information related to the rate of refrigerant delivery, rate of exhaust, temperature, imaging (e.g., from the bronchoscope 160), location information, patient vitals, timers, and/or other information associated with the cryotherapy procedure and/or the patient. The display 112 can be a monitor, touch screen, LCD display, and/or other type of device that communicates information to those associated with the procedure. In some embodiments, the control component 110 is configured to communicate the information associated with the procedure to one or more remote devices (e.g., a laptop, tablet, database) in the procedure room and/or at a remote location (e.g., via Bluetooth, Wi-Fi, ethernet, and the like).

In various embodiments, the control component 110 can generate control signals that control the delivery of refrigerant from the supply component 104, control the operation of the cryotherapeutic device 130, and/or send/receive signals from the cryotherapeutic device 130 and/or other devices associated with the cryotherapeutic procedure (e.g., patient vitals, the bronchoscope 160, sensors) to control other components of the system 100. For example, during a cryotherapy procedure, the control component 110 may control the delivery of refrigerant to the cooling assembly 140 to initiate, maintain, adjust, pause, stop, and/or resume cryotherapeutic cooling to a treatment location of the subject in response to user inputs and/or intraprocedural signals (e.g., detected from a sensor associated with the system 100).

In the embodiment illustrated in FIG. 1A, the console 102 is a singular unit that includes the control component 110 and the supply component 104. For example, the console 102 can include a cart (e.g., including wheels) that carries the control component 110, the supply component 104, and/or other components of the system 100, such that the components can be transported together to suitable locations between and/or during cryotherapy procedures (e.g., different procedure rooms, different locations within a procedure room). In other embodiments, components of the console 102 can be separated from each other. For example, the supply component 104, the control component 110, and/or the display 112 can be operably connected but separated from each other. In some embodiments, the bronchoscope 160 is operably coupled to a separate console and/or separate display from the console 102 such that imaging from the bronchoscope 160 is provided on a separate display.

Referring now to FIGS. 1B and 1C, the cooling assembly 140 can include an expandable member 142, such as a balloon, attached to the distal end portion 136 of the elongated shaft 132 and an injection tube 144 extending from the shaft 132 into the expandable member 142. The injection tube 144 can be in fluid communication with the supply line 106 (FIG. 1A) and the refrigerant supply component 104 (FIG. 1A), via the delivery lumen 133, to deliver refrigerant to the chamber defined by the expandable member 142. For example, the injection tube 144 can include one or more openings 146 (also referred to as “cryo ports”) selectively positioned along the injection tube 144 to expel refrigerant to desired locations within the expandable member 142 and/or select portions thereof. In some embodiments the refrigerant can be delivered via the openings 146 such that it is at a pressure suitable to keep the refrigerant in liquid form (e.g., pressures between 500 psi to 760 psi) until release into the expandable member 142. As shown in FIG. 1C, the interior chamber of the cooling assembly 140 is also in fluid communication with a refrigerant exhaust port 148 at a distal end portion of the exhaust lumen 135 to remove refrigerant from the expandable member 142. In the illustrated embodiment, for example, the exhaust lumen 135 is sized larger than the injection tube 144. The injection tube 144 and the exhaust lumen 135 and associated port 148 may be sized or configured relative to each other based on one or more factors including a desired flow rate and/or pressure of fresh refrigerant to a target site, a desired flow rate and/or pressure of refrigerant exhaust removed from the target site, the extent of expansion of the fresh refrigerant at the target site, combinations thereof, and/or other suitable factors. In some embodiments, for example, the exhaust shaft can have an outer diameter of 2.3 mm to 2.8 mm, smaller than 2.3 mm, or greater than 2.8 mm. In some embodiments, the exhaust lumen 135 can exhaust refrigerant at a high rate (e.g., 3,000-6,000 sccm) to quickly cycle the refrigerant through the expandable member 142. In other embodiments, the exhaust shaft dimensions and/or the exhaust rate may differ. FIGS. 1D and 1E illustrate the flow path of the refrigerant along the cryotherapeutic device 130 in more detail.

In various embodiments, the console 102 can adjust the size (e.g., outer diameter) of the expandable member 142 by inflating the expandable member 142 with more or less pressure to make the expandable member 142 larger or smaller. For example, the expandable member 142 with a maximum outer diameter of 10 mm may be inflated to have an internal pressure of 7 psi (48.26 kPa) during an apposition check (to ensure proper positioning), and then typically run at 3 psi (20.68 kPa) during ablation. However, the console 102 can be configured to increase the pressure and/or decrease the pressure to fit within and accommodate treatment in smaller or larger lumens. Because the expandable member 142 is made of semi-compliant material, it will still take on its fully inflated shape (not misshapen) through a range of pressures (e.g., up to 30 psi (206.84 kPa)). In various embodiments, the console 102 can take in data (e.g., during the apposition check, via sensors on the balloon or other sensors, via inputs from lumen measurements before or during treatment) and adjust the pressure applied to the expandable member 142 to provide appropriate apposition for the target site.

The elongated shaft 132, the delivery lumen 133, the exhaust lumen 135, and/or the injection tube 144 may be made of a material that is substantially inert to the refrigerant, substantially inert to and biocompatible with the physiological environment of the airway, and/or sufficiently flexible at or near the temperature of refrigerant exhaust. In some embodiments, for example, the elongated shaft 132 may be made of a multilayer thermoplastic elastomer material (e.g., thermoplastic polyurethanes, thermoplastic copolyester (e.g., PEBAX)) with braided reinforcement and can have an outer diameter of 0.09 inch (or 2.3 millimeters). The various delivery and exhaust tubes and shafts can also be made from materials sufficiently flexible at or near the temperature of liquid refrigerant or refrigerant exhaust to be delivered to a target site in the airway while sustaining the pressure of the liquid refrigerant or the refrigerant exhaust and the pressure and/or temperature drop when liquid refrigerant is sprayed. For example, the injection tube 144 can be made of Nitinol. In various embodiments, the shaft 132 and/or the injection tube 144 can be made from other suitable materials and/or have different dimensions suitable for delivery to the target site.

In some embodiments, the elongated shaft 132 and/or the handle 138 can include insulative and/or warming elements to enhance stability of the cryoablation in a low heat load environment. To provide cryotherapeutic cooling, the system 100 relies on a balance of (1) refrigerant expansion to gas that creates cooling, (2) the absorption of the cool temperatures (provided by the expanded refrigerant via the surface of the surface of the expandable member 142) by the adjacent tissue, and (3) the evacuation (e.g., via vacuum) of the warmed refrigerant (e.g., nitrous gas warmed due to heat transfer from adjacent tissue) into the exhaust lumen 135. During ablation, parts of the expandable member 142 may not directly contact tissue and are instead exposed to the air within the airways, which does not absorb the cryotherapeutic cooling as quickly, resulting in a low heat load environment along those portions not in direct contact with the tissue. This may cause the system 100 to exhaust the refrigerant (under vacuum) faster than the refrigerant is absorbing/pulling heat from the adjacent tissue. When this occurs, small amounts of liquid refrigerant may not expand within the expandable member 142, causing the liquid refrigerant to enter the exhaust lumen 135, travel proximally toward the handle 138, and, in some instances, expand elsewhere along the proximal portion of the system 100 (e.g., near a flow detector in the console 102), which can cause fluctuations in the flow rate reading. In embodiments where the system 100 is designed to maintain a specific flow rate of the vacuum, these fluctuations in flow rate readings may cause the system 100 to stop the ablation cycle (e.g., display an error message) if there is too much fluctuation in the flow rate. As such, the shaft 132 and/or the handle 138 can include insulating elements and/or conductive heating element to even out the expansion of any remaining liquid refrigerant in the exhaust lumen 135 before it reaches the proximally positioned flow rate detector. For example, the shaft 132 can include an insulating material extending along a portion of its length, such as PEBAX having a greater thickness along a proximal portion of the shaft 132 and/or other suitable insulating materials. The shaft 132 itself can include features that provide an active heat source, such as stainless steel or other conductors extending along a portion of its length such that they can be actively heated. In some embodiments, external warming elements can be applied to the proximal portion of the shaft 132 and/or handle 138. For example, these warming elements can include heat packets, heating pads, heat guns, and/or a warm bath applied along the shaft and/or handle 138 before and/or during ablation. Such insulation and warming elements may be of particular use when the system 100 is used with disposable scopes, which are made of plastic and do not transmit heat to the shaft 132 as well as reusable scopes.

The expandable member 142 can transition between a delivery state and an expanded state (expanded state shown in FIGS. 1B and 1C). In the delivery state, the expandable member 142 may have a low profile (e.g., be deflated) to facilitate its movement through the airways and/or through the working channel of a bronchoscope before and/or after cryotherapeutic cooling. For example, the expandable member 142 can be in the delivery state when extending the cooling assembly 140 beyond the distal end of the bronchoscope 160 (FIG. 1A) until it is in the desired target location for cryotherapeutic cooling (e.g., via delivery of pressurized liquid nitrogen with a low temperature and a high pressure), and/or when retracting the expandable member 142 at least partially back into the distal end of the bronchoscope 160 for removing the cooling assembly 140 from the target site after the treatment is completed.

In the expanded state, the expandable member 142 is inflated (e.g., via a lumen in communication with the interior of the expandable member 142) or otherwise expanded to define an interior chamber suitable for receiving refrigerant. For example, the expandable member 142 can be positioned beyond the distal end of the bronchoscope 160 (FIG. 1A) proximate to the target site and expanded (e.g., inflated via gas, mechanically expanded) to abut and/or press against adjacent tissue of the airway lumen in which it is positioned. When the liquid refrigerant is being delivered from the injection tube 144 into the expandable member 142 via, e.g., one or more openings 146 on the injection tube 144, the refrigerant undergoes rapid expansion due to the pressure drop, thereby transitioning from liquid to gas, and effectuating the rapid cooling of the surrounding tissue in contact with the expandable member 142 for cryotherapeutic neuromodulation. Refrigerant continues to be delivered to within the expandable member 142, and thus the already expanded refrigerant exhausts through the exhaust port 148 to be guided away from the target site via the exhaust lumen 135.

In the inflated configuration, the expandable member 142 can expand radially to conform to the shape of the airway so that, when expanded (e.g., inflated via gas, mechanically expanded) the tissue along the wall of the airway closely contacts the expandable member 142 and is exposed to the cooling from the expanded refrigerant. Furthermore, the expandable member 142 can be sized and shaped such that, when in the expanded state, the expandable member 142 can oppose the inner walls of the adjacent airway section to secure the expandable member 142 in place. For example, the expandable member 142 may be configured to appose the internal wall of the airway circumferentially at a cross section. Further, the expandable member 142 may be made from a compliant and/or semi-compliant material that allows the expandable member to fit within various different airway sizes while also conforming at least partially to an interior wall of an airway so as to press against or abut the tissue. For example, the expandable member 142 can be sized to have a maximum outer diameter in the expanded state of 10 mm-15 mm and/or other diameters therebetween. In some embodiments, the expandable member 142 with a maximum diameter of 10 mm to 15 mm can be used to adequately press against and treat airway lumens that have a diameter of 12-15 mm, which is the typical size of the left upper lobe bronchus and the right intermediate bronchus. In some embodiments, the expandable members 142 with an outer diameter of 10 mm has been used to effectively treat lumens with an average size of 5 mm and as small as 4.1 mm (e.g., the size of some right upper lobe bronchus). In some embodiments, the expandable member can treat smaller or larger airway lumens. The expandable member 142 can also be made from a material that provides for and can withstand rapid heat transfer to allow for the cryotherapeutic cooling of adjacent and surrounding tissue. The material may be substantially inert to and biocompatible with the physiological environment of the airway, and/or sustain the temperature of the refrigerant expelled from the injection tube 144 at which cryotherapy proceeds. For example, the expandable member 142 may be made of polyurethane, polyethylene, polytetrafluoroethylene (PTFE), polyvinyl chloride (PVC), nylon, PEBAX, silicone, polyimide, polypropylene, or the like, or a combination thereof.

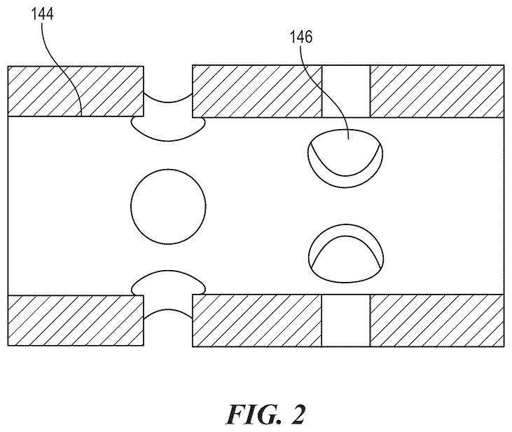

The one or more openings 146 along the injection tube 144 can be located in select positions along the length and/or circumference of the injection tube 144 to provide for a desired cryoablation pattern. For example, the openings 146 can be oriented in a manner that creates one or more fully circumferential lesions that are 360° at a cross section of the airway wall. This cryoablation pattern may be further enhanced by delivering the refrigerant from the injection tube 144 substantially uniformly in the circumferential direction along a desired cross section of the airway wall. This may be achieved by, for example, arranging openings 146 circumferentially in a uniform pattern in one or more rows (e.g., two rows, three rows, more than three rows) around the injection tube 144. FIG. 2, for example, provides an example of openings 146 arranged in two rows around the injection tube 144, in which the individual openings 146 in adjacent rows are offset from each other to provide for a unform cooling pattern along a fully circumferential cross-section of the expandable member 142 (FIG. 1C). As described in further detail below, in other embodiments, the openings 146 are oriented and/or selectively activated in a manner that provides cryoablation along a non-fully circumferential cross section of the expandable member 142 and the adjacent airway it is configured to treat. For example, the openings 146 can be oriented or activated in a manner that provide for cooling along a hemispherical portion of the expandable member 142 (and adjacent airways) and/or creates cooling regions in spaced apart zones along a length or radial portion of the expandable member 142.

As another example and as described in further embodiments below, when denervation via cryoablation involves creating one or more lesions that are less than 360° at any cross section, the expandable member 142 may exhibit a non-occlusive cross section such that the cross-section of the expandable member 142 touches only a portion of a circumferential segment of the internal wall at any cross section of the airway. For example, the expandable member 142 may have non-occlusive and eccentric cross sections configured to form ablation of a linear shape, a curved shape, or a spiral shape, over a segment of the airway. The non-occlusive expandable member 142 may allow lung venting, clearing mucus, or positioning a device (e.g., a guidewire, a mucus clearing device), or the like, or a combination thereof, when the expandable member 142 is at the inflated configuration.

In some embodiments, the injection tube 144 may include an external tube and a delivery sheath where the refrigerant delivery holes 146 are located. The delivery sheath may extend beyond or retract into the injection tube 144. The number (or count) of the refrigerant delivery holes 146 operable to dispel refrigerant to the interior of the expandable member 142 may be adjusted by adjusting the length of the delivery sheath extending beyond the external tube of the injection tube 144. In some embodiments, the delivery sheath may be rotatable such that the direction at which the refrigerant is sprayed into the expandable member 142 may be adjusted by rotating the delivery sheath. The adjustment (e.g., rotation, extension, or retraction) of the delivery sheath may be controlled or implemented under the control of the handle 138 (FIG. 1B) and/or manually by a user by manipulating a portion of the cryotherapeutic device 130.

Referring again to FIG. 1C, the cooling assembly 140 can include an atraumatic tip portion 150 that is rounded or otherwise shaped to provide an atraumatic leading surface of the cooling assembly 140. The tip portion 150 can also or alternatively provide a support structure to which a distal end portion of the injection tube 144 and/or the distal end portion of the expandable member 142 are supported or otherwise secured.

In some embodiments, the cooling assembly 140 can include one or more sensors 154 that detect one or more parameters associated with the cooling assembly 140. The one or more sensors 154 can be positioned along or within the injection tube 144, at the exhaust port 148 and/or within the exhaust lumen 135, along the expandable member 142, and/or at other locations within or on the cooling assembly 140. For example, the sensor 154 can be a thermocouple that measures the temperature within the expandable member 142 and/or along the wall of the expandable member 142. This information can be communicated to the console 102 (FIG. 1A) such that the operator can track the temperature and confirm the desired cooling temperatures are met. In some embodiments, the sensor 154 may measure pressure within the expandable member 142, the pressure of the expandable member 142 against an adjacent airway wall, the flow rate of the refrigerant exhaust, the flow rate of the refrigerant in the injection tube 144, and/or other parameters.

In various embodiments, the cooling assembly 140 can further include a valve, e.g., a needle valve, a Joule-Thomson throttle, a throttle element, or the like, for providing a pressure drop and a lower downstream temperature, thereby alter and/or enhance the effect and/or efficiency of the cryotherapeutic ablation. For example, a Joule-Thomson throttle can recover work energy from the expansion of the refrigerant resulting in a lower downstream temperature.

As further shown in FIG. 1C, the cooling assembly 140 can include one or more radiopaque markers 152 positioned along the injection tube 144 to allow for location guidance. In other embodiments, the radiopaque markers 152 can be positioned elsewhere on the cooling assembly 140, such as on the expandable member 142 and/or along the shaft 132.

As further shown in FIGS. 1B and 1F, the cryotherapeutic device 130 can further include a balloon protector component 137 (also referred to as a “cooling assembly protector,” a “protector assembly,” an “introducer sheath,” and the like) along the shaft 132 and configured to extend over the expandable member 142 to protect (e.g., provide a barrier, encase, cover) the components of the cooling assembly 140 as it moves into and out an inlet of the working channel 162 the bronchoscope 160 and/or before a procedure while the cooling assembly 140 is packaged and stored). The protector component 137 can be a tubular structure that slidably extends over a portion of the shaft 132 and is sized to extend over at least a portion the cooling assembly 140. In some embodiments, a portion of the protector component 137 (e.g., a distal portion) is sized greater than the inlet of the bronchoscope working channel 162 such that, when the distal portion of the shaft 132 is threaded into the working channel 162, the protector component 137 abuts the surface adjacent the working channel 162 and does not extend therein. The protector component 137 can be made of a material having at least some rigidity to provide a protective covering for the cooling assembly 140 and/or provide additional rigidity to a portion of the shaft 132. During a procedure, the protector component 137 can initially be in a position at least partially over the cooling assembly 140 with a distal-most end extending to or beyond the distal-most end of the cooling assembly 140. When the distal portion of the shaft 132 with the cooling assembly 140 is threaded through the working channel 162 of the bronchoscope 160, the protector component 137 protects the cooling assembly 140 as it enters the working channel 162 (e.g., avoids damage to the balloon at the inlet) and/or facilitates insertion into the inlet of the working channel 162. As the shaft 132 continues to be threaded distally through the working channel 162, the protective component 137 stops at least partially outside the working channel 162, allowing the shaft 132 to slide therethrough. The protector component 137 can further impart stiffness along a portion of the shaft 132 immediately proximal to the working channel 162 to facilitate insertion into the bronchoscope 160. Therefore, the protector component 137 can reduce or eliminate the risk of kinks in the shaft 132 when manipulating the shaft 132 through tortuous anatomy during tracking. As further shown in FIG. 1F, a proximal portion of the protective component 137 can include a flared proximal region (e.g., an inlet) that flares radially outwardly or otherwise defines an opening larger in cross-sectional dimension (e.g., diameter) than that of the distal or medial portion so as to ease insertion of the cooling assembly 140 into the protective component 162.

Referring to FIGS. 1A-1F together, the system 100 is configured to affect tissue at a depth beyond the tissue which it contacts so as to cause a therapeutic effect on structures, such as nerves, positioned a distance away from the tissue in contact with the cooling assembly 140. This “ablation zone,” imparts thermal injury to neural fibers a distance from the bronchial lumen. The distance from the bronchial lumen and size of the ablation zone can depend, at least in part, on the ablation pattern provided by the cooling assembly 140, the duration of ablation, and the number of cycles of ablation. For example, the system 100 can ablate neural fibers positioned 1 mm-3 mm from the inner wall of a bronchial lumen. In some embodiments, the system 100 can impart thermal injury to neural fibers more than 3 mm from the inner wall of the bronchial lumen. In some embodiments, the thermal injury can be acute and/or the injury occurs a time period after the initial ablation procedure (e.g., 7-30 days after). In some embodiments, the thermal injury to certain portions of the affected region (e.g., select tissue) heal after a period of time, whereas other portions, such as neural fibers, can be completed partially or ablated. The targeted lung denervation performed with the system 100 has been shown denervate the bronchial branches of the vagal nerve to reduce hyperreactivity. This is expected to reduce airway constriction, inflammation, and/or mucus production. The denervation of bronchial branches of the vagal nerve also is expected to produce a therapeutic effect in COPD patients including, e.g., reduced occurrence or severity of exacerbations, an altered course of decline in a Pulmonary Function Test (PFT), and/or improvement in the quality of life of a patient.

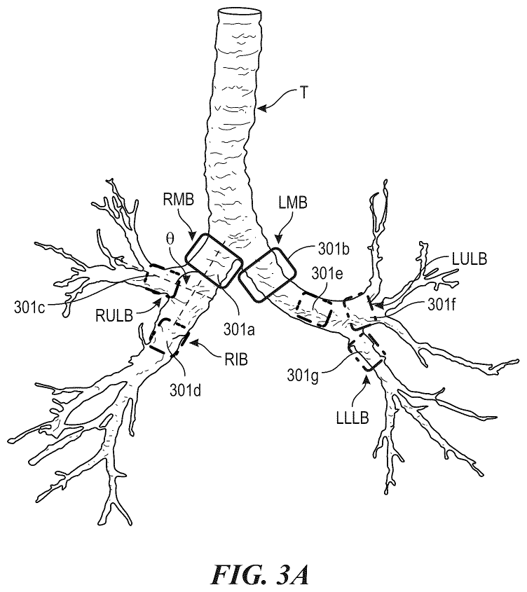

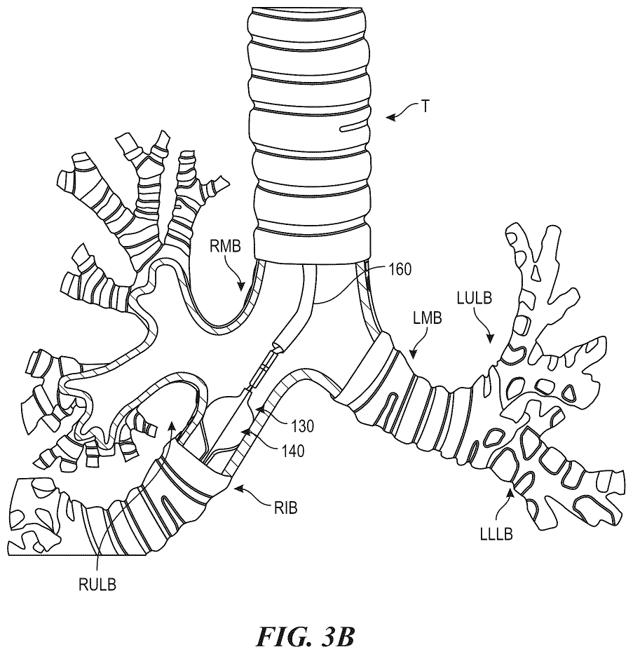

FIG. 3A illustrates airway anatomy with exemplary treatment locations for a cryotherapy procedure in accordance with some embodiments of the technology, and FIG. 3B illustrates the distal portion of the cryotherapeutic device 130 of FIG. 1B performing an ablation procedure at a treatment location in an airway in accordance with embodiments of the present technology. Referring to FIGS. 3A and 3B together, the airway anatomy includes a trachea T that branches into a right main bronchus RMB and a left main bronchus LMB. The right main bronchus RMB further branches into smaller bronchi and bronchioles within the lung tissue in the right lung, including the right upper lobe bronchus RULB and right intermediate bronchus RIB. The left main bronchus LMB further branches into smaller bronchi and bronchioles within the lung tissue in the left lung, including the left upper lobe bronchus LULB and left lower lobar bronchus LLLB. As shown in FIG. 3A, treatment sites 301 (identified individually as first through seventh treatment sites 301a-g, respectively) can be positioned within the right main bronchus RMB and the left main bronchus LMB, as well as distally therefrom beyond the bifurcations, within the right and left upper lobe bronchus RULB, LULB, the right intermediate bronchus RIB, and the left lower lobar bronchus LLLB. As such, cryotherapeutic devices disclosed herein, such as the device 130 of FIG. 1A, have shafts and end effectors that are sized, shaped, and configured to navigate through tortuous airway anatomy and into the airways beyond the main right and left bronchus RMB, LMB, which have smaller diameters. For example, an angle between an axis of the right main bronchus RMB and the right upper lobe bronchus RULB, referred to the right upper lobe bronchus angle θ, can be an obtuse angle that requires a shaft of a cryotherapeutic device (e.g., the device 130 of FIGS. 1A-1C) to navigate a substantial curvature to reach any target sites within right upper lobe bronchus RULB. Accordingly, to reach a treatment location in the airway distal to the bifurcation from the right main bronchus RMB and the right upper lobe bronchus RULB, the shaft of the cryotherapeutic device 130 (FIGS. 1A-1C) must be able to flex or otherwise bend by an angle that is substantially the same as or similar to the right upper lobe bronchus angle O. The ability to treat distal to the main bifurcation and bifurcations beyond the main right and left bronchus allows the present technology to avoid important anatomy (e.g., blood vessels, nerves not associated with the present indication) so as to avoid the cryotherapeutic cooling from adversely impacting the nearby structures.

As discussed above, the cryotherapeutic devices disclosed herein may be sized, shaped, and configured for use with a bronchoscope to navigate and visualize a cryotherapy procedure directed at a treatment location. The positioning of the distal portion of the cryotherapeutic device, or a portion thereof (e.g., an end effector), may be delivered and visualized via the bronchoscope, thereby allowing the user to precisely navigate and position the catheter within the airway. This can facilitate accurate targeting of a specific area in the airway that needs treatment. In addition, the bronchoscope may provide real-time visualization of the airway during the procedure, thereby allowing the user and/or the system to monitor the treatment site and make an adjustment if needed to ensure effective treatment and minimizing damage to surrounding tissues. For example, based on the visuals provided by the bronchoscope, the user (e.g., a clinician) may reposition the end effector to a suitable target site and/or to better oppose the adjacent airway wall, adjust the degree of inflation of the end effector to suitable anchor and/or oppose the adjacent airway wall, and/or adjust a parameter of the refrigerant delivery (e.g., the pressure and/or a flow rate of fresh refrigerant to the cryotherapeutic device or refrigerant exhaust from the cryotherapeutic device, an injection direction of fresh refrigerant exiting the cryotherapeutic device via a nozzle, refrigerant flow duration, a time interval between freezing and thawing in an ablation cycle, or the like, or a combination thereof). In some embodiments, the catheter shaft may contain markers along the length of its distal portion for positioning guidance visualization via the bronchoscope.

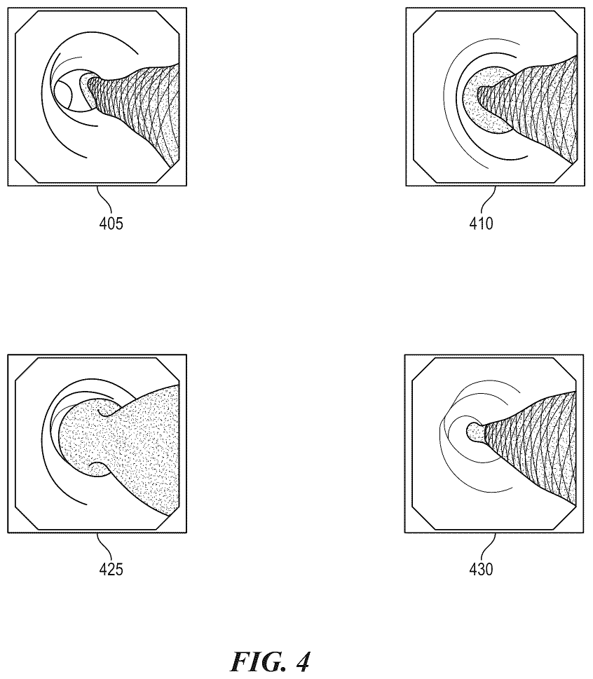

FIG. 4 illustrates bronchoscopic images of a cryotherapeutic procedure using a cryotherapeutic catheter (e.g., the cryotherapeutic device 130 of FIGS. 1A-1C) configured in accordance with embodiments of the present technology. The procedure can begin by first advancing a bronchoscope (e.g., the bronchoscope 160 of FIG. 1A) toward a treatment site within the bronchus (e.g., within the right or left branch, beyond the bifurcation of the right or left branch). The distal end portion of the cryotherapeutic device can then be inserted into the bronchoscope's working channel (e.g., while being supported by the balloon protector assembly 137 of FIG. 1B) and threaded therethrough such that a cooling assembly (e.g., the cooling assembly 140 of FIGS. 1A-1B) projects beyond the distal opening of the working channel and can be visualized as shown in step 405. The expandable member can then, optionally, be inflated (e.g., with air or other gas) to confirm the exterior surface of the expandable member provides appropriate apposition against the tissue of the inner wall of the airway. Although the cooling assembly with the semi-compliant balloon is designed to adequately appose and treat a large range of airway diameters, if the expandable member does not provide adequate apposition, e.g., because it is either too large or too small, the catheter can be removed, and a differently sized balloon can be used. After confirming appropriate apposition, the expandable member can be deflated and, optionally, retracted into the bronchoscope to allow the bronchoscope to capture one or more images of the treatment site. In some procedures, this step is omitted.

Once the clinician is ready to initiate cryotherapeutic cooling, the cooling assembly can be moved to the target location and the position of the cooling assembly can be confirmed (e.g., via the radiopaque markers, by viewing images/video via the bronchoscope, and/or other suitable location confirmation mechanisms). The bronchoscope handle (at the proximal end of the bronchoscope) may be attached to the bronchoscope via attachment features (e.g., clips, interfacing components, snap arms, on the handle), and the bronchoscope can optionally be positioned in a bronchoscope holder such that the bronchoscope together with the handle remain stationary during the procedure without being held by the clinician. As shown in step 410, the expandable member can then be inflated such that it presses against the tissue at the target location and the delivery of refrigerant for cryotherapeutic neuromodulation can be initiated. In some embodiments, the breath of the patient is held immediately before balloon inflation and for the first portion of the ablation such that there is little to no movement at the target site, and thereby maintaining the position of the expandable member at the target site.

As shown in step 425, initiation of the ablation step (i.e., by delivering the refrigerant to the cooling assembly) can cause ice to form around the exterior of the expandable member. This freezes the expandable member to the surrounding tissue and stabilizes the position of the expandable member relative to the target site. In some embodiments, the cryotherapeutic cooling provided by the systems disclosed herein can cause an ice ball to form around an entire circumference of the expandable member and the abutting airway wall, or a portion thereof. With the balloon adhered or otherwise secured at the target site (e.g., via the ice ball), the patient breath hold can be released. The delivery of cryotherapeutic cooling can continue (e.g., with or without breath holds) until the desired cryotherapeutic cycle of refrigerant delivery has run. Upon completion of the cryotherapeutic cooling, the procedure can continue by deflating the balloon and capturing an image of the treatment site (step 430) and/or taking measurements of the treatment site.

In some embodiments, the cryotherapeutic cooling procedure can be performed multiple times at the same target site (e.g., the balloon deflated and then reinflated for one or more additional ablation cycles). In some embodiments, the cryotherapeutic device provides for partial circumferential ablation, and therefore the catheter can be rotated to ablate other portions along the circumference of the airways.

Combining the cryotherapeutic catheter with the bronchoscope may result in a less invasive procedure compared to traditional surgical methods, which may lead to reduced trauma to the treatment site and/or the surrounding tissues, shorter recovery times, reduced pain, and minimized risk of complications. The flexible nature of both the bronchoscope and cryotherapeutic device may allow access to remote or hard-to-reach areas of the airways, such as the distal treatment locations in the bronchi and other branches of the airway illustrated in FIG. 3A.

In addition to delivering cryotherapeutic cooling, the cryotherapeutic device may be configured to serve one or more various other purposes including, e.g., delivery of pharmaceuticals (e.g., analgesics, local anesthesia medicines), fluids, and/or therapies directly to the target site, suction of mucus or debris, detection of data surrounding the target site, thereby allowing for tailored treatments based on the patient's condition and needs.

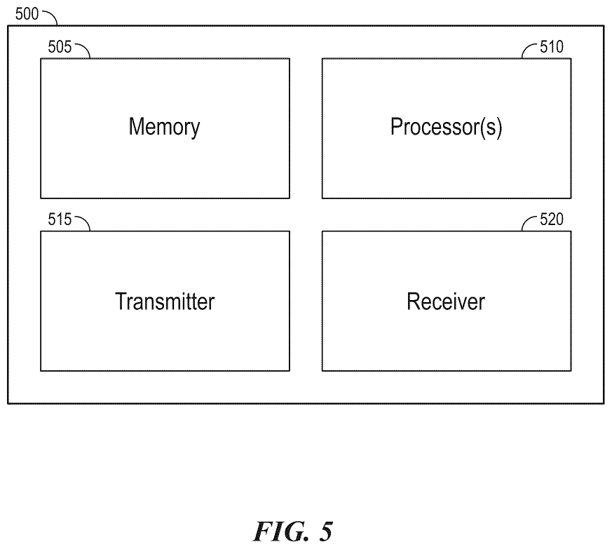

FIG. 5 illustrates a block diagram of a processing device configured to perform control of the system 100 in accordance with some embodiments of the technology. The processing device 500 may constitute an example of the control component 110 (FIG. 1A). The processing device 500 may be operably coupled to or integrated with a cryo console, a display, input devices, output devices, and/or other devices or components of cryotherapeutic system (e.g., features of the system 100 of FIG. 1A). The processing device 500 may be configured to receive information, data, signals, and/or other feedback, and generate control signals to control the operation of the system 100 of FIG. 1A and components thereof.

The processing device 500 may include memory 505 and processor(s) 510. The memory 505 may have instructions stored thereupon. The instructions, upon execution by the processor(s) 510, may configure the processing device 500 (e.g., the various modules or components of the processing device 500) to perform the operations described elsewhere in the present document including. The processor(s) 510 may include at least one processor and/or control circuitry to send and receive commands, requests, and other suitable data using one or more input/output (I/O) paths.

The control circuitry may include any suitable processing, storage, and/or I/O circuitry. The processing device 500 may also include or be operably connected to a user input interface and/or user output interface. As referred to herein, a “user interface” may include a human-computer interaction and communication in a device and may include display screens for use in receiving and displaying data, input devices (e.g., a keyboard, a touchscreen, a mouse), and the appearance of a desktop. For example, a user interface may include a way a user interacts with the control components and/or the intraprocedural monitoring components.

In some embodiments, the processing device 500 may include a transmitter 515 and a receiver 520 configured to send and receive information, respectively. At least one of the transmitter 515 or the receiver 520 may facilitate communication via a wired connection and/or a wireless connection between the processing device 500 and a device or information resource external to the processing device 500. For example, the processing device 500 may receive sensor data acquired by sensors of an intraprocedural monitoring component via the receiver 520. As another example, the processing device 400 may receive input from a user via the receiver 520. As a further example, the processing device 500 may transmit a notification to a user (e.g., a medical professional, a display) via the transmitter 515. In some embodiments, the transmitter 515 and the receiver 520 may be integrated into one communication device.

### Select Embodiments of Injection Tubes for Refrigerant Delivery

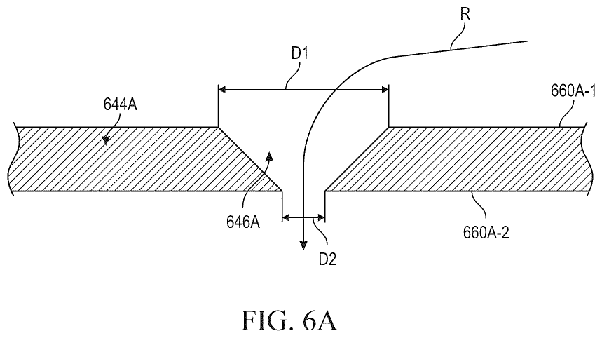

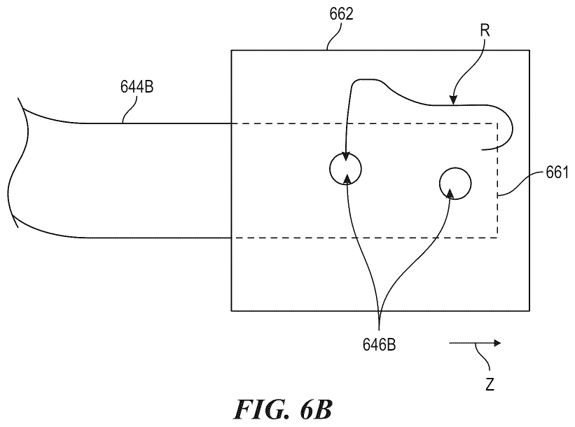

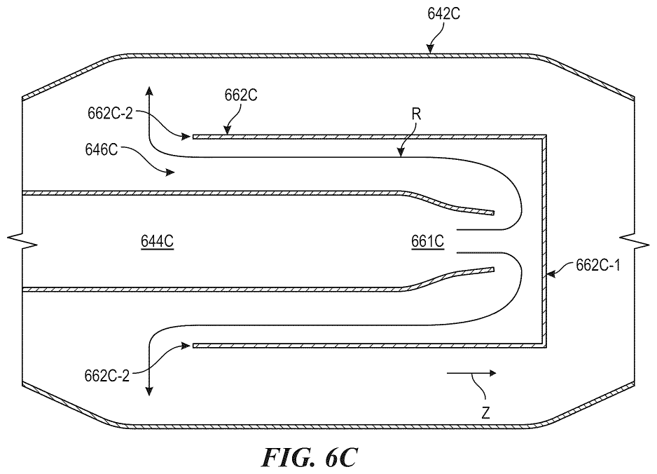

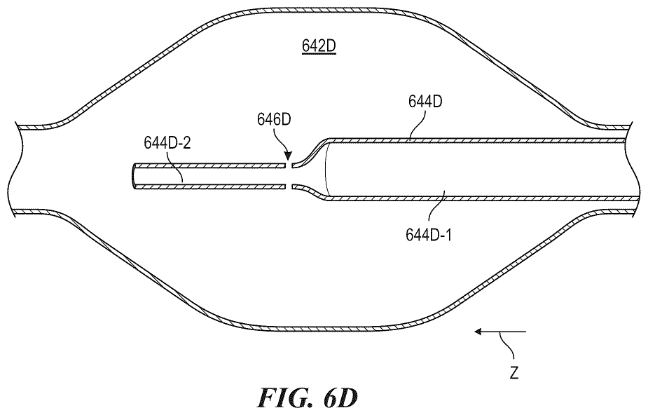

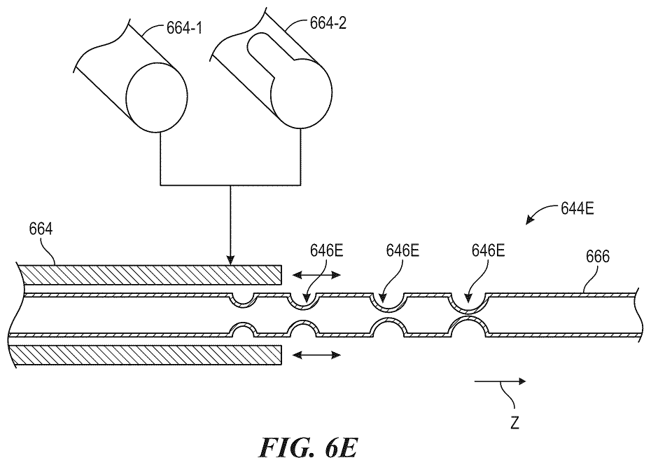

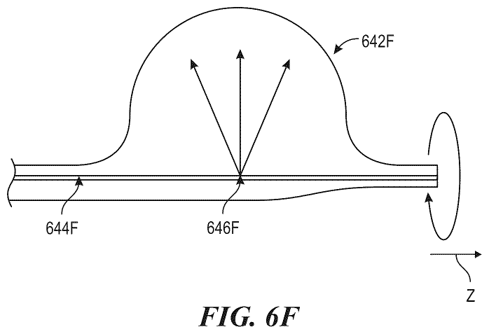

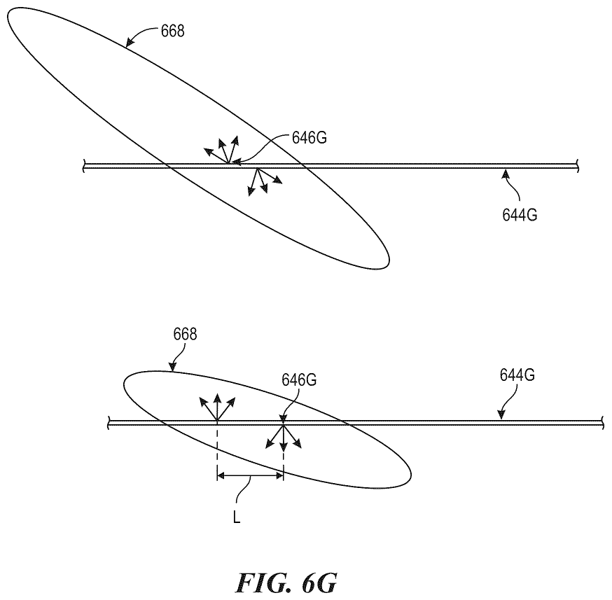

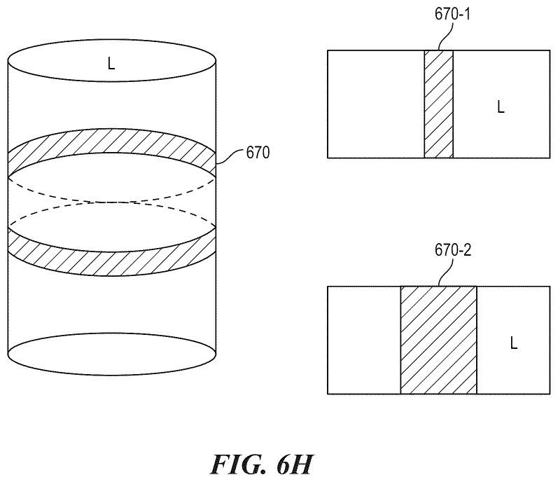

FIGS. 6A-6I illustrate features of injection tubes for cryotherapeutic devices (e.g., the cryotherapeutic device 130 of FIGS. 1A-1C) in accordance with some embodiments of the technology. For example, cryotherapeutic devices configured in accordance with embodiments of the present technology can include injection tubes that are configured to pressurize refrigerant immediately before expansion to enhance the Joule-Thompson effect of gas expansion through a small orifice into the relatively large enclosure formed by an expandable member (e.g., the expandable member 142 of FIGS. 1B and 1C). This enhances the cooling effect of the refrigerant as it enters into the expandable member and improves the efficiency of heat transfer between the expanded refrigerant and bronchial tissue in contact with and/or near the expandable member. In some embodiments, an injection tube may be configured to facilitate a directional injection and/or spray of refrigerant to provide a targeted and focused thermal profile along a surface of the expandable member, thereby enhancing the efficiency of heat transfer between the refrigerant and surrounding tissue and reducing the cooling to other regions of tissue contacting the expandable member. This can reduce the impact of cryotherapeutic cooling on tissue and/or structures located near the target site. In some embodiments, the features of the injection tubes and/or associated ports for expelling refrigerant described below can be combined.

FIG. 6A is a cross-sectional side view of an injection tube 644A for a cryotherapeutic device (e.g., the cryotherapeutic device 130 of FIGS. 1A-1C) configured in accordance with embodiments of the present technology. The injection tube 644A includes at least one an opening 646A (also referred to as a cryoport or refrigerant outlet) for expelling pressurized refrigerant (illustrated by arrow R) from the injection tube 644A into an expandable member (e.g., a balloon, the expandable member 142 of FIGS. 1B and 1C) to effectuate cryotherapeutic cooling. As shown in FIG. 6A, the opening 646A is wedge-shaped and tapers between an inner surface 660A-1 of the tube 644A to an outer surface 660A-2 of the tube 644A. Thus, the opening 646A has a larger dimension D1 (e.g., diameter if the opening 646A has the shape of a circle) at the inner surface 660A-1 (e.g., at the inner diameter) of the injection tube 644A than a dimension D2 of the opening 646A at the outer surface 660A-2 (e.g., at the outer diameter) of the injection tube 644A. In the illustrated embodiment, the opening 646A has a constant taper from the inner surface 660A-1 to the outer surface 660A-2. However, in other embodiments, the opening 646A can be stepped, curved, and/or otherwise shaped to be narrowing at the outlet of the opening 646A than the outlet. The narrowing of the opening 646A toward the outer surface 660A-2 of the injection tube 644A increases the pressure of the refrigerant immediately before the refrigerant expands into the expandable member, thereby increasing the pressure differential experienced by the refrigerant upon entry into the expandable member. This increased pressure differential enhances the cooling caused by rapid expansion of the refrigerant and can thereby provide enhanced cryotherapeutic cooling. FIG. 6A illustrates one narrowed opening 646A, but it will be appreciated that the injection tube 644A can include two or more narrowed openings 646A.

FIG. 6B is a side view of an injection tube 644B for a cryotherapeutic device (e.g., the cryotherapeutic device 130 of FIGS. 1A-1C) configured in accordance with embodiments of the present technology. As shown in FIG. 6B, the injection tube 644B has an outlet 661 at its distal end region, and a collar 662 (also referred to as a cap or covering member) that extends over the outlet 661 and is sealed to the distal end portion the injection tube 644B (e.g., along the proximal end of the collar 662. The collar 662 may be fully or partially sealed to the injection tube 644B. The collar 662 has one or more openings 646B (also referred to as a cryo ports or refrigerant outlets) along its sidewall and in fluid communication with the outlet 661 to allow pressurized refrigerant from the injection tube 644B to expel (e.g., spray) into an expandable member (e.g., a balloon, the expandable member 142 of FIGS. 1B and 1C) to effectuate cryotherapeutic cooling. During a cryotherapeutic procedure, the refrigerant releases from the outlet 661, into the space between the outside of the injection tube 644B and the interior of the collar 662, and exits through the openings 646B of the collar 662. The openings 646B of the collar 662 are sized and shaped to create ports with a smaller area than the outlet 661 for refrigerant to be emitted (e.g., sprayed) into the surrounding expandable member. This embodiment is suitable in embodiments where the injection tube 644B terminates within the expandable member. (e.g., does not extend along its entire length). In embodiments where the collar is partially sealed to the injection tube 644B, there is one or more openings defined by the proximal end of the collar and the injection tube 644B such that refrigerant may be expelled in the proximal direction. In other embodiments, the outlet 661 is positioned along the sidewall of the injection tube 644B and the collar 662 extends over the portion of the injection tube 664B with the outlet 661 to provide small ports that provide an increased pressure differential (in comparison to the outlet 661) for emitting refrigerant.

FIG. 6C is a cross-sectional side view of an injection tube 644C for a cryotherapeutic device (e.g., the cryotherapeutic device 130 of FIGS. 1A-1C) configured in accordance with embodiments of the present technology. Similar to the embodiment of FIG. 6B, the assembly includes a cap 662C that extends over an outlet 661C at the distal end portion of the injection tube 644C. The cap 662C is closed at the distal end 662C-1 that opposes the outlet 661C and open at a proximal end portion 662C-2 to an expandable member 642C (e.g., a balloon). One or more gaps between the proximal open end 662C-2 of the cap 662C and the injection tube 644C defines one or more openings 646C sized to expel refrigerant into the expandable member 642C. As shown via the fluid path R, the refrigerant that leaves the outlet 661C into the distal end 662C-1 of the cap 662C, where it turns to flow in a proximal direction, where it can exit into the expandable member 642C via the one or more openings 646C. The sizing of the opening(s) 646C and associated fluid pathway defined by the gap between the outer surface of the injection tube 644C and the inner surface of the cap 662C can be selected based on the relative sizing of the two components to provide a desired pressure differential as the refrigerant exits the opening(s) 646C into the chamber defined by the expandable member 642C. In some embodiments, the outlet 661C has a diameter less than a diameter of the rest of the injection tube 644C. By decreasing the diameter at the outlet 661C, the refrigerant may be expelled at a higher pressure. The change in diameter of the injection tube 644C may be similar to that described below.

FIG. 6D is a side view of an injection tube 644D within an expandable member 642D at a distal end portion of a cryotherapeutic device (e.g., the cryotherapeutic device 130 of FIGS. 1A-1C) in accordance with embodiments of the present technology. As shown in FIG. 6D, the injection tube 644D has a first portion 644D-1 with a first dimension (e.g., inner diameter) and a second portion 644D-2 with a second dimension (e.g., inner diameter) different than the first dimension. In the illustrated embodiment, the first portion 644D-1 is positioned proximal to the second portion 644D-2, and the first portion 644D-1 has a larger dimension (e.g., diameter) that transitions to a smaller dimension along the second portion 644D-2 at or near where cryoports 646D are located. However, the cryoports 646D may be located along the first portion 644D-1, the second portion 644D-2, at a transition between the portions, or a combination thereof. Such a configuration further pressurizes refrigerant at the distal end portion immediate before expansion into the expandable member 642D, thereby enhancing the Joule-Thompson effect and resultant cooling. A change in diameter between the first portion 644D-1 and the second portion 644D-2 may be linear where the diameter decreases at a constant rate, curvilinear where the diameter decreases nonlinearly (e.g. quadratic, spline, etc.), stepped where the sections are separated by a straight section, or other methods.

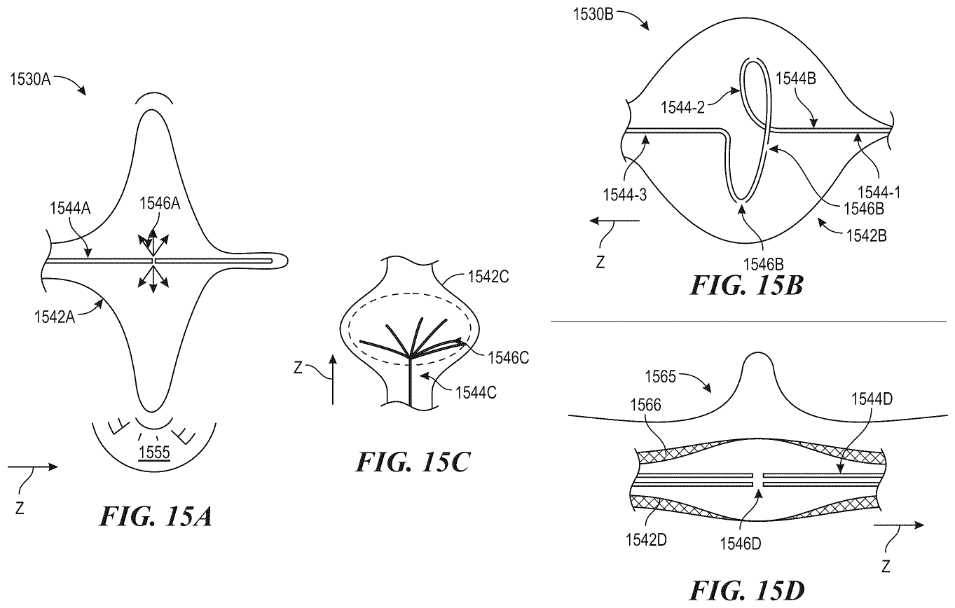

The injection tubes described herein may include one or more additional or alternative features configured to increase the pressure profile of refrigerant. For example, a proximal portion of an injection tube may include a single lumen, and that single lumen may divide into or transition to multiple secondary lumens along a distal portion of the injection tube at or near where cryoports are located. See, for example, FIG. 15C, and relevant description thereof. As another example, cryoports may be biased toward a distal end of an injection tube, in combination with a surrounding balloon of a sufficient volume, to maintain an overall volume for expansion to achieve Joule-Thompson cooling effect.

FIG. 6E illustrates a cross-sectional side view of an injection tube assembly 644E configured in accordance with embodiments of the present technology. The injection tube assembly 644E includes a sheath 664 extending at least partially over an inner tube 666 along which one or more cryoports 646E (openings) are located. The cryoports may be located along a length of the inner tube 666, around a radius of the inner tube 666, or a combination thereof. The sheath 664 and the inner tube 666 may slide, rotate, or otherwise move relative to each other along or against the direction of the Z-axis to expose a desired number (or count) of cryoports 646E. The relative movement between the sheath 664 and the inner tube 666 may be controlled via an actuator operably coupled to the sheath 664 and/or the inner tube 666. The actuator may be disposed on a handle of a cryotherapeutic device (e.g. handle 138 of device 130). This allows the user to adjust the number of cryoports 646E that emit refrigerant into a surrounding balloon to provide the desired cooling effect. For example, the sheath 664 may have a sidewall that is fully circumferential along its length (see, e.g., sheath 664-1) so that the sheath 664 may cover or expose cryoports 646E circumferentially when sliding (e.g., in a distal direction) relative to the inner tube 666. As another example, the sheath 664 may have one or more cutouts (e.g., open regions) along its sidewall (see, e.g., sheath 664-2), allowing for specific functionalities or access points, so that the sheath 664-2 may expose cryoports 646E that are aligned with the cutout(s) along the sheath 664-2 and cryoports 646E distal to the distal terminus of the sheath 664-2. In this embodiment, the direction at which refrigerant is injected or sprayed from the injection tube 644E into a surrounding balloon chamber, and the region of the balloon and opposing tissue that receives cooling, may be controlled or adjusted by rotating the sheath 664-2.

FIG. 6F is a side view of an injection tube 644F and an expandable member 642F of a cryotherapeutic device (e.g., the cryotherapeutic device 130 of FIGS. 1A-1C) in accordance with embodiments of the present technology. As shown in FIG. 6F, the injection tube 644F can be oriented toward one side of the expandable member 642F and/or include cryoports 646F oriented on one side of the injection tube 644F to direct refrigerant into the chamber defined by the expandable member 642F. In some embodiments, the expandable member 642F is asymmetric such that a first side of the expandable member 642F may be expanded to a larger diameter than an opposing side of the expandable member 642F. In some embodiments, the injection tube 644F can be positioned within the expandable member 642F and the end effector (e.g., the expandable member 642F in conjunction with the injection tube 644F) may be rotated within the airways to adjust the injection direction of refrigerant relative to the airways. This rotation can be initiated by rotating the entire catheter shaft or via an actuator (e.g., on a proximal handle) that can rotate the distal portion of the catheter shaft. In some embodiments, the injection tube 644F itself can be rotated within an expandable 642F (e.g., via an actuator coupled to the injection tube 644A or manual rotation) to adjust the injection direction of the refrigerant. In such embodiments, the expandable member 642F may be asymmetric.

FIG. 6G illustrates schematic side views of injection tubes 644G for cryotherapeutic devices (e.g., the cryotherapeutic device 130 of FIGS. 1A-1C) configured in accordance with embodiments of the present technology. In the illustrated embodiment, the injection tubes 644G include cryoports 646B that are longitudinally offset from each other by a distance L along the longitudinal axis of the injection tube 644G and that are arranged on opposing sides (e.g., 180 degrees apart from each other) of the injection tube 644G. This offset arrangement of the cryoports 646G creates an elliptical cooling profile 668 along an expandable member and the tissue that it abuts. That is, rather than a cooling profile that extends circumferentially along a cross-section taken perpendicular to the lumen (bronchus), the elliptical cooling profile 668 can be circumferential or partially-circumferential along a diagonal plane of the bronchus, thereby preserving bronchial tissue along radially adjacent portions of the bronchus. As seen in comparing the injection tubes 644G, a greater distance L may cause a major axis of the elliptical cooling profile 668 to increase relative to a minor axis. In some embodiments, one or more cryoports 646G may be located along the elliptical cooling profile 668 between the opposing cryoports. In some other embodiments, the cryoports 646G can be arranged in a manner along the injection tube 644G to create a spiral cooling profile. In such embodiments, the cryoports 646G may follow a helical path with a fixed or variable pitch and may be arranged at a fixed or variable spacing between each other.

FIG. 6H illustrates partially transparent isometric and side views of a therapeutically cooled region 670 along a body lumen L, such as a bronchus, treated using a cryotherapeutic system configured in accordance with embodiments of the present technology. As described herein, an injection tube (e.g., the injection tubes described herein) can emit refrigerant within an expandable member (e.g., a balloon) to provide a cooling profile across a region of the expandable member and adjacent tissue to therapeutically cool the tissue to provide neuromodulation. As shown in the isometric view of FIG. 6H, the cooled region 670 can extend around a fully circumferential segment of the lumen L. This can be provided, for example, by multiple cryoports (e.g., 5 openings) radially spaced apart from each other in a row around a perimeter (e.g., a circumference) of the injection tube. The width of the cooled region 670 can be modified, for example, by the number of rows of openings around the injection tube and/or a size of the openings. For example, referring to the side views of FIG. 6H, lumen L may have a first cooled region 670-1 having a first width when cooled via a cryotherapeutic device having a single row of refrigerant openings, and the lumen L may have a second cooled region 670-2 having a second width greater than the first width when cooled via a cryotherapeutic device having two adjacent rows of refrigerant openings. Depending upon the target site, a narrower or wider cooled region may be desired to treat a greater portion of tissue at once or provide for narrow cooled regions that avoid adjacent structures.

FIG. 6I is a partially cross-sectional side view of a distal portion of a cryotherapeutic device 630I configured in accordance with embodiments of the present technology. As shown in FIG. 61, the device 630I can include a delivery shaft 636I, an expandable member balloon 642I (e.g., a balloon) fluidly connected to the distal end portion of the delivery shaft 636I, an injection tube 644I extending beyond the delivery shaft 636I at least partially into the expandable member 642I, and an exhaust lumen 635I. The injection tube 644I includes at least one opening 646I and a rotary device 672 configured to rotate while emitting refrigerant circumferentially via the one or more openings 646I into the expandable member 642I. Emitted refrigerant absorbs heat from tissue adjacent the surface of the expandable member 642I (thereby cooling the adjacent tissue), and the heated refrigerant moves out of the expandable member 642I via the exhaust lumen 635I. The exhaust lumen 635I may be substantially the same as or similar to the exhaust lumen as described herein.

The rotary device 670 may be a passive rotary turbine manifold driven by liquid refrigerant moving through the manifold's blades, thereby obviating the need for a separate power source to drive the turbine. This will cause the one or more openings 646I to rotate to provide for circumferential cooling along the adjacent tissue (e.g., despite only having a single opening or openings that are not fully circumferential) and may provide for immediate coalescence of the cooling profile and thus substantially uniform circumferential treatment. In some embodiments, the device 630I may have other features that rotate the opening(s) 646I. For example, the entire injection tube 644I can rotate via a motor coupled thereto, manual movement thereof via a handle actuator, or another method. The injection tube 644I can include other rotatable components (e.g., a bushing) that can rotate upon refrigerant emission.

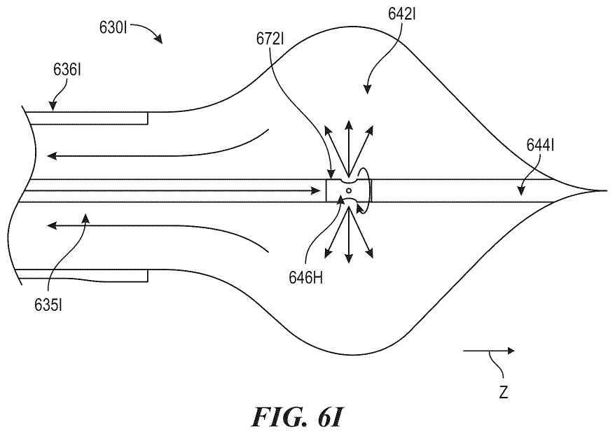

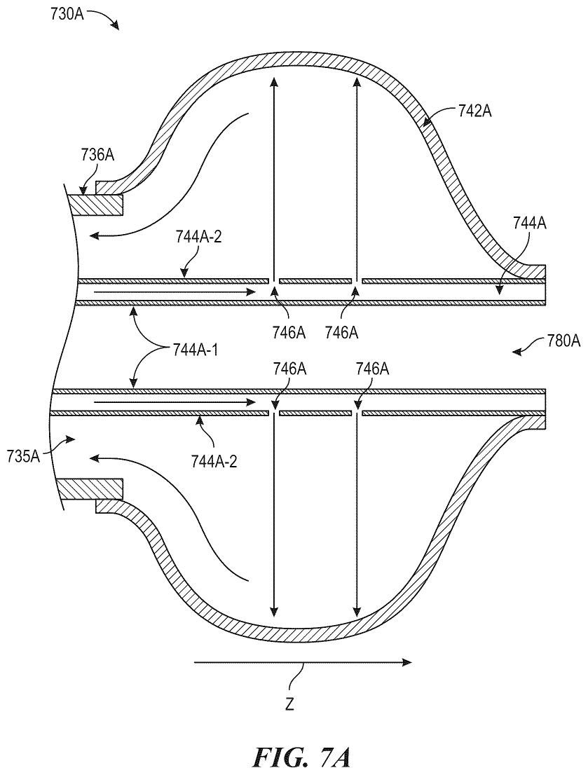

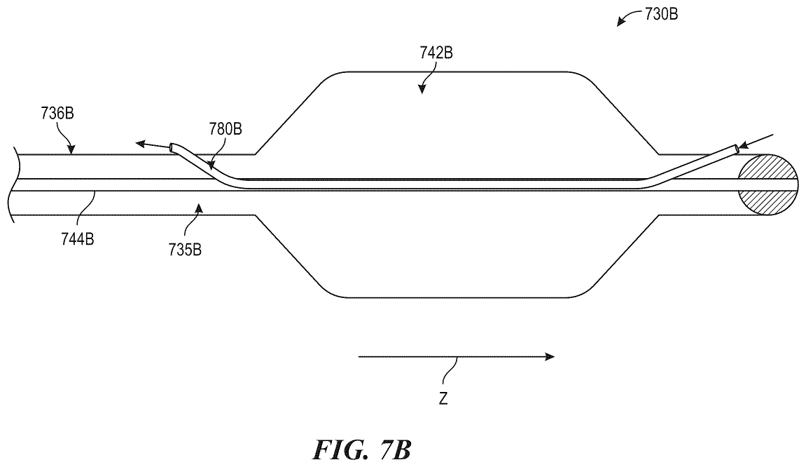

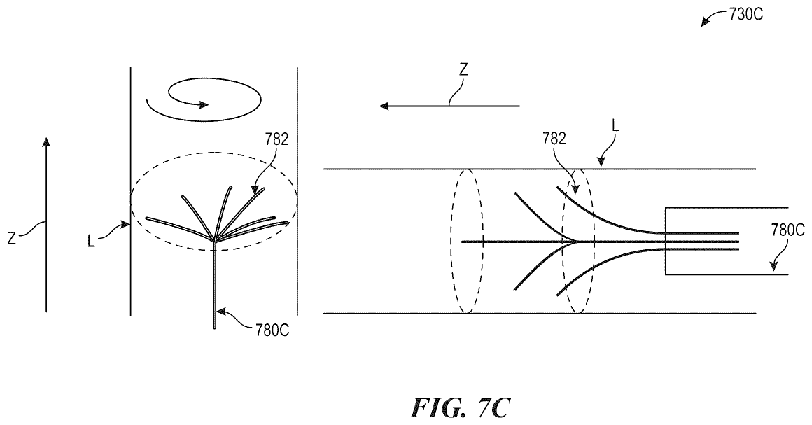

FIGS. 7A-7C are side views of distal portions of cryotherapeutic devices with catheter ports and/or ancillary lumens configured in accordance with some embodiments of the present technology. The devices may have various features and functions of the cryotherapeutic devices described herein and may be sized to fit within a lumen of an endoscope (e.g., the bronchoscope) such that the distal portion thereof may be delivered to or near the target site via the endoscope (e.g., the lumen of the endoscope). The devices of FIGS. 7A-7C further include an additional lumen and/or associated catheter port configured for one or more functions other than refrigerant delivery/exhaust. For example, the additional lumen can provide a channel to avoid complete airway lumen obstruction during cryotherapeutic procedures and/or provide a channel for delivering a guidewire or other tool into the airway. For example, the additional lumen can provide for lung venting, mucus clearance, delivery of tools that allow for in vivo measurements (e.g., measurement of a dimension of a body lumen at or near the target site, temperature measurements), electrophysiology (“EP”) mapping (e.g., via electrodes or other EP device to confirm relative positioning before a procedure and/or effectiveness of a procedure), tissue manipulation (e.g., cutters), and/or supplemental therapies (e.g., injection of therapeutic agents into the bronchial tissue).

FIG. 7A is a partially cross-sectional side view of a distal portion of a cryotherapeutic device 730A (“device 730A”) configured in accordance with some embodiments of the technology. The device 730A may include a delivery shaft 736A, a balloon 742A (or other expandable member) attached to a distal end of the delivery shaft 736A, an injection tube 744A in fluid communication with the balloon 742A via one or more openings 746A, and an exhaust lumen 735A through which refrigerant exhaust may be guided away the balloon 742A (e.g., to an exhaust reservoir).

The device 730A of FIG. 7A may be substantially the same as or similar to the cryotherapeutic devices described above, except the addition of the ancillary lumen 780A and for the configuration of the injection tube 744A. In the embodiment illustrated in FIG. 7A, the injection tube 744A has an inner wall 744A-1 and an outer wall 744A-2, and the inner wall 744A-1 (or a portion thereof) defines a channel having an annular cross section. As illustrated in FIG. 7A, outer wall 744A-2 of the injection tube 744A includes the one or more openings 746A that face interior of the balloon 742A to emit refrigerant into the balloon 742A. The channel created by the inner wall 744A-1 can define or otherwise provide for the ancillary lumen 780A that extends through the injection tube 744A, the balloon 742A, and along at least a portion of the catheter shaft 736A of the device 730A. In some embodiments, the ancillary lumen 780A extends to a handle operably connected to the catheter shaft. The ancillary lumen 780A may be substantially concentric to the delivery shaft 736A as shown in FIG. 7A or offset to one side of the delivery shaft 736A. The ancillary lumen 780A is separate from and not in fluid communication with the balloon 742A, the injection tube 744A, and/or the exhaust lumen 735A, and can provide access to and/or from the lumen (e.g., the airway) into which it is inserted. For example, the ancillary lumen 780A may be open to and/or in fluid communication with the airway into which it is inserted, thereby allowing for lung venting, mucus clearing, and avoiding airway obstruction during a cryotherapeutic procedure (e.g., when the balloon 742A is expanded). The ancillary lumen 780A may also or alternatively provide a lumen for a guidewire such that the device 730A can be delivered with or without a bronchoscope, and/or provide for the delivery of other tools to the target site within the airway (e.g., measuring devices, thermal sensors, injection devices for delivering therapeutics, suction devices for removing mucus). In various embodiments, the distal end of the ancillary lumen 780A may include a protector (e.g., an atraumatic tip, an atraumatic coating) to provide an atraumatic distal end of the device 730A as it is advanced through the airways and/or other body lumen.

FIG. 7B is a partially cross-sectional side view of a distal portion of a cryotherapeutic device 730B (“device 730B”) configured in accordance with some embodiments of the technology. The device 730B may include a delivery shaft 736B, a balloon 742B (or other expandable member) attached to a distal end of the delivery shaft 736B, an injection tube 744B in fluid communication with the balloon 742B via one or more openings (not shown), and an exhaust lumen 735B through which refrigerant exhaust may be guided away the balloon 742B (e.g., to an exhaust reservoir). The device 730B of FIG. 7B can include substantially similar features as the cryotherapeutic devices described herein, except that the device 730B includes the ancillary lumen 780B formed by a bypass tube. The bypass tube extends from a side port accessible via the outer surface of the delivery shaft 736B, into the balloon 742B (e.g., where it may extend alongside the injection tube 744B) and terminate at an exit port along the distal end of the device 730B (e.g., at the side or end of the distal tip). The ancillary lumen 780B can provide similar functions as the ancillary lumens described above, such as guidewire and/or tool delivery.

FIG. 7C illustrates partially schematic front view (left) and side view (right) of a distal portion of a cryotherapeutic device 730C (“device 730C”) configured in accordance with some embodiments of the technology. The device 730C includes an ancillary lumen 780C that accommodates one or more tubes 782. The tube(s) 782 can be positioned within the ancillary lumen 780C when the device 730C is delivered to and/or removed from the target site and, at the target site, can be moved (e.g., via an actuator and/or manual movement via a proximal handle) out of a distal opening of the ancillary lumen 780C. The tubes 782 can be used to emit (e.g., spray, dispel) a cryoprotectant (e.g., a cryoprotectant gel) onto portions of an inner wall of the body lumen L (e.g., the inner wall of the bronchus) to form a mask along the wall to protect the tissue from a subsequent cryotherapeutic cooling treatment (e.g., regions surrounding the target site to protect nearby structures, such as the epithelium) and/or provide a landing pad for the subsequently expanded balloon of the device 730C. In some embodiments, the tubes 782 that dispel cryoprotectant on nearby tissue may be fluidly connected to at least one of the other tubes 782. As seen in the image to the right of FIG. 7C, the tubes 782 may be independent of each other, the tubes 782 may split into multiple tubes (e.g., with multiple lumens) toward the distal end portion, and/or a combination thereof. In some embodiments, the cryotherapeutic device 730C can be coupled to a fluid source having something other than a cryoprotectant fluid source, such as a therapeutic agent, such that one or more of the tubes 782 can dispel a substance other than a cryoprotectant on tissue at or proximate to the target site.

The ancillary lumens described herein may be configured to establish a path for fluid communication between various portions of the body lumen (e.g., an airway) that are otherwise separated or blocked (e.g., by an inflated balloon of the cryotherapeutic device). For example, the ancillary lumen may allow fluid (e.g., gas, liquid including mucus) to bypass the inflated balloon so as to facilitate/achieve one or more of lung venting or mucus clearance. The ancillary lumen may allow a mucus clearing device to access mucus in a portion of the airway near where the balloon apposes the internal wall of the airway and/or to enhance the suction capacity of the mucus clearing device). The ancillary lumen may allow space for a delivery channel to deliver cryoprotectant to a desired location (e.g., a bronchus surface) before the balloon of the catheter expands so that the cryoprotectant may act as a mask or landing pad for the balloon when it expands to its inflated configuration and apposes the internal wall of a body of the object.

In various embodiments, the ancillary lumens may allow space for a guidewire, a delivery channel, or a measurement component. For example, the ancillary lumen may accommodate a guidewire configured to assist an endoscope (e.g., the bronchoscope 160) in delivery of a balloon of the catheter to challenging anatomy. As another example, the ancillary lumen may allow space for placing a measurement component (e.g., a flexible guidewire or pigtail) configured to measure the internal diameter (“ID”) of the body lumen (e.g., a bronchus). This may obviate the need to prescreen patients. As a further example, the ancillary lumen may allow space for placing an electrophysiology (EP) mapping catheter for EP mapping of bronchi so as to combine EP mapping of the bronchi and esophagus for confirming the relative position of the distal portion of the cryotherapeutic device, the cooling zone of the end effector, and/or target nerves before proceeding with the application of refrigerant to or near the target site. This may allow for monitoring proximity of a distal portion of the cryotherapeutic device (where the refrigerant application is effectuated) to the esophagus and ensure/improve procedural safety and/or to confirm the target treatment area.

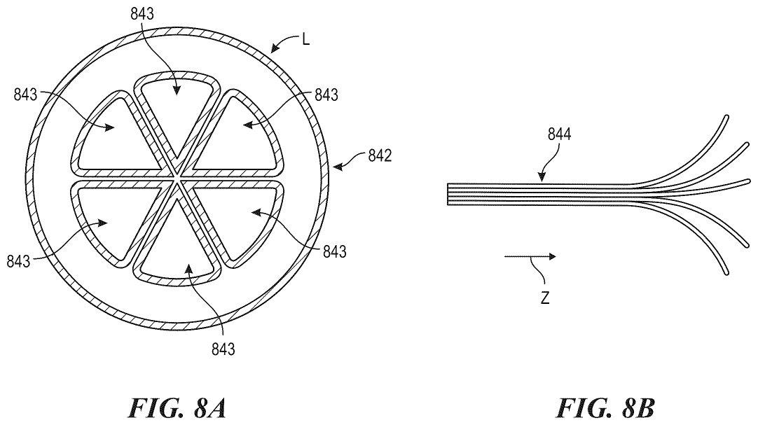

In some embodiments, cryotherapeutic devices described herein include expandable members, such as balloons, which provide a non-occlusive cross-section in the expanded (e.g., inflated) configuration such that the cross-section of the expandable member touches only a portion of a circumferential segment of the internal wall at any cross-section of the airway. FIGS. 8A-13 illustrate various designs for expandable members (e.g., balloons) that provide a non-occlusive cross-section in the expanded state in accordance with some embodiments of the technology.

FIGS. 8A and 8B are a cross-sectional end view of an expandable member 842 (also referred to as “the balloon 842”) and a side view of a corresponding injection tube 844 in accordance with some embodiments of the technology, respectively. The balloon 842 may have multiple lobes that leave open lumens 843 (also referred to as passageways and open regions) therebetween when expanded. In some embodiments, one or more lobes of the balloon 842 are fluidically coupled to one another. In other embodiments, one or more lobes of the balloon are fluidically isolated from each other. FIG. 8A illustrates the balloon 842 in an inflated state. When fully inflated, the balloon may appose an internal wall L of a body lumen (e.g., an airway), thereby allowing air or other fluid to flow through the open regions 843 between the lobes of the balloon 842. This prevents the body lumen (e.g., bronchus) in which the balloon 842 is positioned from being completely blocked by the inflated balloon 842 and fluid (e.g., gas, mucus) may flow past the inflated balloon 842 (along or against the direction of the axis Z) during a cryotherapeutic procedure.

In some embodiments, two or more of the lobes of the balloon 842 may receive refrigerant from separate branches of an injection tube 844 and, in some embodiments, the lobes are fluidly isolated from each other. As illustrated in FIG. 8B, the injection tube 842 may include multiple branches, each of which may be configured to feed refrigerant to a lobe of the balloon 842. In some embodiments, at least a portion of the lumens of the balloon 842 may be in fluid communication with each other such that these lobes may receive refrigerant from the same branch of the injection tube 844.

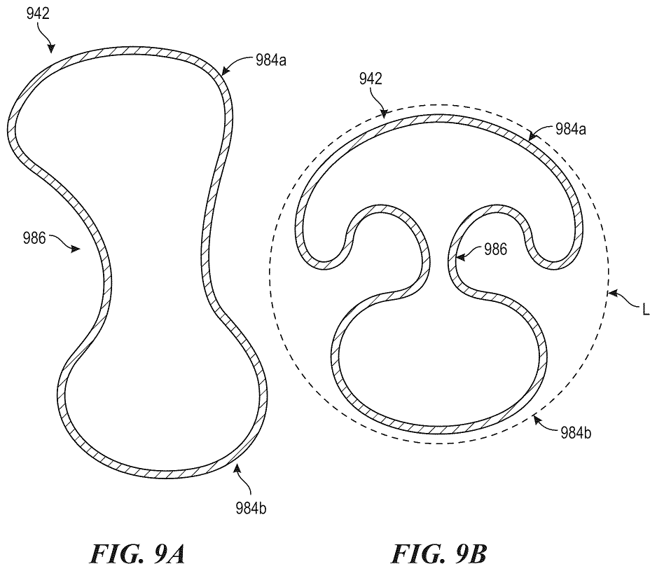

FIG. 9A is cross-sectional end-on view of a non-occlusive expandable member 942 (also referred to as “the balloon 942”) configured in accordance with some embodiments of the technology, and FIG. 9B is a cross-sectional end-on view of the expandable member 942 positioned in an airway lumen L. The expandable member 942 has two lobe portions 984 (identified individually as a first lobe 984a and a second lobe 984b) and a middle portion 986 (also referred to as a medial portion or an interconnecting portion) positioned between and connecting the two lobe portions 984. The middle portion 986 can be smaller (e.g., in diameter) than the two lobe portions 984. As shown in FIG. 9B, when expanded within the airway lumen L, the two lobe portions 984 of the balloon 942 appose and press against sections of inner wall of the airway lumen L, but the middle portion 986 does not appose the internal wall of the airway lumen L, thereby creating open lumens through which fluid (e.g., gas, mucus) may flow past the inflated balloon 942 (along or against the direction of the axis Z) during a cryotherapeutic procedure. In some embodiments, the balloon 942 may include more than two lobe portions 984 with medial portions 986 extending between each of the lobe portions 984. The middle portions 986 and lobe portions 984 may be fluidically coupled to one another, fluidically isolated to one another, or a combination thereof. In some embodiments, an injection tube may split into branches that may feed into the middle portions 986 and/or the individual lobe portions 984 to focus cryogenic cooling in the respective region.

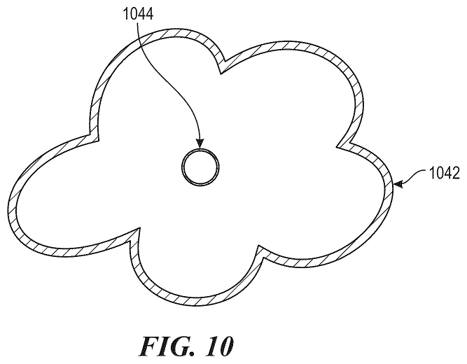

FIG. 10 illustrates an end-on cross-sectional view of an expandable member 1042 (also referred to as “the balloon 1044”) configured in accordance with some embodiments of the technology. As shown in FIG. 10, the cross-section of the balloon 1042 has a floral or cloud-like shape with multiple small indented sections 1043 (also referred to recessed regions and the like) positioned around the exterior of the balloon 1044 to define multiple lobes 1045 having larger outer diameters in an expanded state than the indented sections 1043. Various portions of the balloon 1042 (e.g., the lobes 1045), when inflated, may appose discontinuous portions of an internal wall of a body lumen (e.g., a bronchus) and the indented sections 1043 can prevent the body lumen from being completely blocked by the inflated balloon 1042 (e.g., to allow for airflow during cryotherapeutic treatment). The balloon 1042 may have a single injection tube 1044 that delivers refrigerant from a single, central location. In other embodiments, the injection tube 1044 can include multiple branches that deliver refrigerant to different portions of the balloon 1042. In some embodiments, the indented sections 1043 may be formed from locally thickened portions of a flexible membrane of the expandable member 1042 and/or other materials that limit expansion of the expandable member 1042 at the indented sections 1043. In some embodiments, the expandable member may be shaped with indented sections 1043 during a manufacturing process (e.g. blow molding into contoured cavity, etc.). In some embodiments, the indented sections 1043 may be formed by applying ribs externally or internally to the expandable member 1042. The ribs may be made with a semi-compliant material.

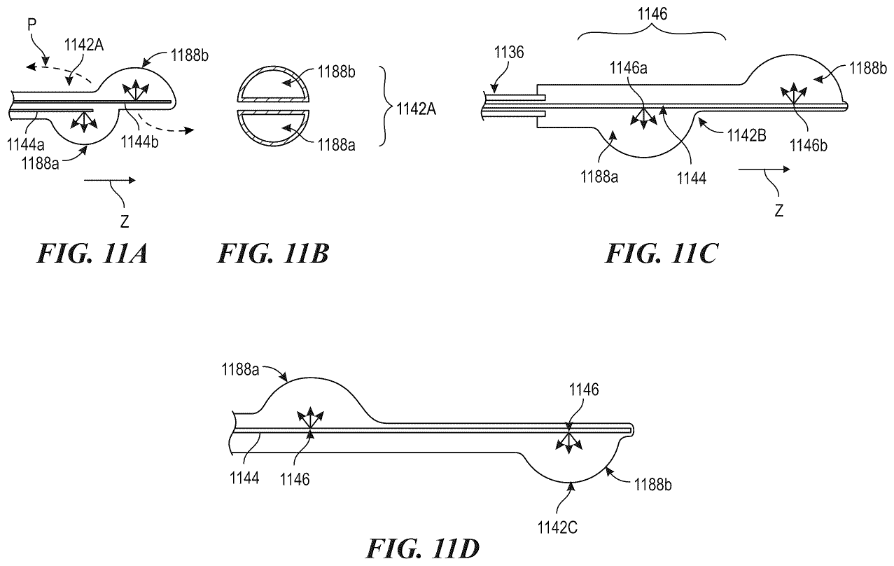

FIGS. 11A-11D illustrate side and a cross-sectional views of various expandable members (e.g., balloons) for cryotherapeutic devices configured in accordance with some embodiments of the technology. FIG. 11A is a side view of a balloon 1142A that has a first lobe 1188a that projects in a first direction toward a first portion of an internal wall of a body lumen on one side of a longitudinal axis (e.g. substantially parallel to the axis Z) of the balloon 1142A, and a second lobe 1188b that projects in a second direction (e.g., opposite the first direction) toward a second portion of the internal wall of a body lumen on another side of the longitudinal axis (e.g. substantially parallel to the axis Z) of the balloon 1142A. The first lobe 1188a and the second lobe 1188b of the balloon 1142A are spaced longitudinally apart from each other along the longitudinal axis of the device (e.g., along the Z-axis). Accordingly, the balloon 1142A, when expanded, provides a flow path P that gas, mucus, or other fluid can flow during a cryotherapeutic procedure and avoid full occlusion of the airway. In some embodiments, the first lobe 1188a and the second lobe 1188 b are positioned in opposite directions such that they are rotated 180 degrees from one another. However, in other embodiments, the lobes may be positioned such that they positioned from 0 to 360 degrees from one another while spaced longitudinally apart. The lobes 1188 may be substantially hemispherical (e.g., as shown in FIGS. 11A-11D), sub-hemispherical, super-hemispherical, and/or in an irregular shape.

As further shown in FIG. 11A, the first lobe 1188a can be supplied refrigerant via a corresponding first injection tube 1144a, and the second lobe 1188b can be supplied refrigerant via a separate second injection tube 1144b. In some embodiments, the two lobes 1188 fluidly isolated from each other. In other embodiments, the two lobes 1188 are in fluid communication with one another, but are supplied with refrigerant from the two corresponding injection tubes 1144. In some embodiments, the first lobe 1188a and the second section 1188b of the balloon 1142A may be spaced apart and positioned on either side of an anatomical structure that the user does not want to expose to cryotherapy (e.g., a cartilage ring along the body lumen). In such embodiments, the balloon 1142A does not appose the portion of the internal lumen wall corresponding to the anatomical structure (e.g., the cartilage ring) and the anatomical structure is protected from cryoablation.

The injection tubes 1144 may include one or more openings positioned to expel refrigerant into the expandable member 1142A. In some embodiments, a first one or more openings are located on the first injection tube 1144a such that the refrigerant is expelled in a direction of the first lobe 1188a and a second one or more openings are located on the second injection tube 1144b such that the refrigerant is expelled in a direction of the second lobe 1188b. The openings may be arranged in a variety of ways along the injection tubes 1144 to define a desired longitudinal range and/or angular range of the refrigerant's expel pattern suitable for the corresponding portion of the expandable member 1142A. For example, the first one or more openings may be arranged in one or more hemispherical rows on the first injection tube 1144a to align with the portion of the first lobe 1188a. In another example, the first one or more openings may arranged on the first injection tube 1144a such that each opening may be at a same angular position but located with a longitudinal spacing corresponding to a length of the first lobe 1188a. Other arrangements are possible and may be chosen based on a position of the lobes, the shape of the lobes, and/or a desired ablation pattern. The openings of the first injection tube 1144a may be arranged similarly to or different from the openings of the second injection tube.

FIG. 11B illustrates a cross-sectional view of the balloon 1142A of FIG. 11A along the Z-axis showing that the two lobes 1188 can provide for fully circumferential ablation, but along longitudinally spaced apart regions of the body lumen. In other embodiments, the two lobes 1188 are arranged such that they do not provide for fully circumferential ablation.

FIG. 11C is a side view of a balloon 1142B that, similar to the balloon 1142A of FIG. 11A, has a first lobe 1188a that projects in a first direction toward a first portion of an internal wall of a body lumen on one side of a longitudinal axis (substantially parallel to the axis Z) of the balloon 1142B, and a second lobe 1188b that projects in a second direction (e.g., opposite the first direction) toward a second portion of the internal wall of a body lumen on another side of the longitudinal axis (substantially parallel to the axis Z) of the balloon 1142B. The first lobe 1188a and the second lobe 1188b of the balloon 1142A are spaced longitudinally apart from each other along the longitudinal axis of the device (e.g., along the Z-axis). The balloon 1142B may be securely and/or hermitically connected to a delivery shaft 1136. In the embodiment illustrated in FIG. 11C, both lobes 1188 of the balloon 1142B are supplied refrigerant via the same injection tube 1144, and the injection tube 1144 may have one or more first openings 1146a that emits refrigerant into the first lobe 1188a, and have one or more second openings 1146b that emits refrigerant into the second lobe 1188b. In some embodiments, the injection tube 1144 may have separate lumens extending therethrough corresponding each of the first openings 1146a and the second openings 1146b. The openings 1146 may be oriented in different direction corresponding to the positions of the lobes 1188 such that refrigerant is sprayed substantially toward the corresponding lobe 1188 and/or in a direction where various target sites are located. For example, the first opening 1146a and the second opening 1146b may be oriented 180 degrees apart from each other, which may create a partially circumferential or an elliptical thermal profile on the tissue that opposes the balloon 1142.

FIG. 11D is a side view of a balloon 1142C that has a first lobe 1188a that projects in a first direction on one side of a longitudinal axis (substantially parallel to the axis Z) of the balloon 1142C, and a second lobe 1188b that projects in a second direction (e.g., opposite the first direction) toward another side of the longitudinal axis (substantially parallel to the axis Z) of the balloon 1142C. Similar to the that the balloon 1142B of FIG. 11C, the lobes 1188 are supplied by a single injection tube 1144. However, the lobes 1188 are elongated in comparison to those of FIG. 11C and thus can treat a larger segment of the bronchus. One or more openings 1146 may be located on the single injection tube 1144. In some embodiments, there may be a first and second set of the one or more openings 1146 positioned such that the first one or more openings 1146 may supply refrigerant to the first lobe 1188a and the second one or more openings 1146 may supply refrigerant to the second lobe 1188a. The arrangement of the one or more openings 1146 may be substantially similar to that described above.

In the embodiments illustrated in FIG. 11A-11D, the expandable member 1142 is shown and described as having two lobes 1188. In other embodiments, an expandable member may include more than two lobes 1188 that are spaced longitudinally apart from one another along the longitudinal axis of the distal portion of the cryotherapeutic device. In such embodiments, there may be one or more injection tubes that are configured to supply refrigerant to the at least three lobes. The lobes may be fluidly isolated from one another, fluidly coupled to one another, or a combination thereof. For example, an expandable member includes four lobes that project in four directions at 90-degree intervals from one another (e.g., spaced 90 degrees apart from each other along the longitudinal axis of the device). In some embodiments, all four lobes are supplied by the same injection tube with openings directing fluid in each of the lobes). In other embodiments, two or more injection tubes can supply refrigerant to the lobes. There may be a wide variety of other arrangements of lobes, injection tubes, and openings where projection angle, form, longitudinal spacing, and other variables are adjusted based on desired ablation patterns.

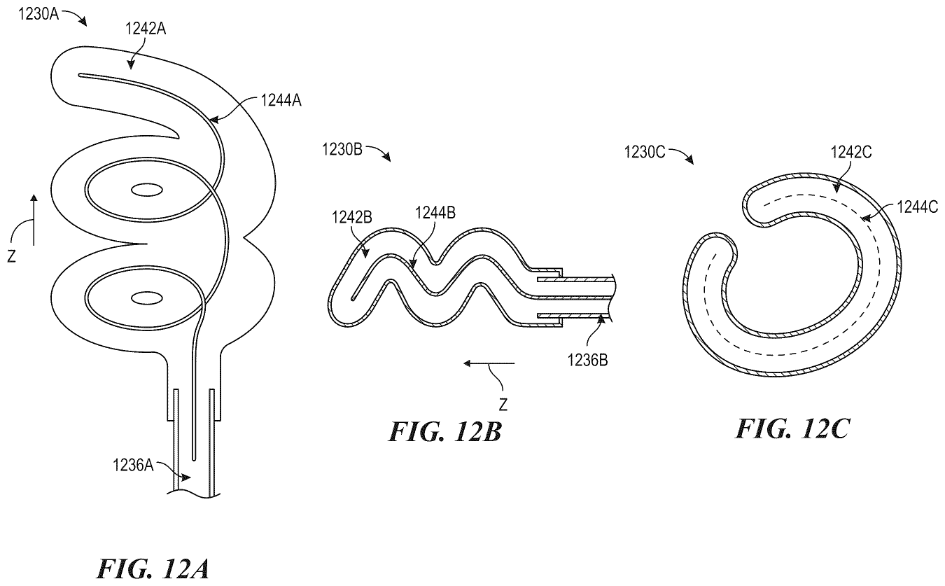

FIGS. 12A-12C illustrate schematics of side views and a cross-sectional view of distal portions of various cryotherapeutic devices configured in accordance with some embodiments of the technology. FIG. 12A illustrates a cryotherapeutic device 1230A including a delivery shaft 1236A, an expandable member balloon 1242A (also referred to as “the balloon 1242A”) sealed to the delivery shaft 1236A, and an injection tube 1244A. The balloon 1242A may, when inflated, have a spiral shape so as to appose various discontinuous portions of an internal wall of a body lumen (e.g., an airway). The injection tube 1244A may have a spiral shape that is similar to or generally follows the shape of the balloon 1242A, be substantially concentric with the balloon 1242A, and/or extend through a substantial portion along the length of the balloon 1242A. The balloon 1242A and the spiral injection tube 1244A may have variable pitch and/or diameter. The spiral injection tube 1244A may create a spiral thermal profile within the tissue at or near where the balloon 1242A apposes the internal wall. The thermal profile in the tissue may substantially correspond to where cryoablation occurs. In other embodiments, the balloon 1242A may have other suitable shapes (e.g., hemispherical, helical, amorphous) that are configured to appose discontinuous segments of a lumen wall.

FIG. 12B illustrates a cryotherapeutic device 1230B including a delivery shaft 1236B, an expandable member 1242B (also referred to as “the balloon 1242B”) coupled to the delivery shaft 1236B, and an injection tube 1244B. The balloon 1242B, when inflated, may have a wave-like shape so as to appose various discontinuous portions of an internal wall of a body lumen in an object. The injection tube 1244B may have a similar wave-like shape that corresponds to the shape of the balloon 1242B, be substantially concentric with the balloon 1242B, and/or extend through a substantial portion along the length of the balloon 1242B.

FIG. 12C illustrates a cryotherapeutic device 1230C including an expandable member 1242C (also referred to as “the balloon 1242C”) and an injection tube 1244C extending therein. The balloon 1242C has a toroidal shape that is not fully circumferential (e.g., a C-like shape). The injection tube 1244C may have a shape that is similar to the shape of the balloon 1242C, be substantially concentric with the balloon 1242C, and/or extend through a substantial portion along the length of the balloon 1242C. In some embodiments, the injection tube 1244B may include multiple tubes and/or branched lumens positioned around the toroidal shaped balloon 1242 C.

An articulating balloon as illustrated in FIGS. 12A-12C may be employed to traverse a complex anatomy (e.g., characterized by a varying or small dimension, a large turning angle at a bifurcation, and/or tortuous nature of a body lumen) to deliver cryotherapy at or near a target site. If desired, such an articulating balloon may be straightened partially or completely (e.g., after approaching the target site) by increasing the pressure in the balloon or by straightening the injection tube.

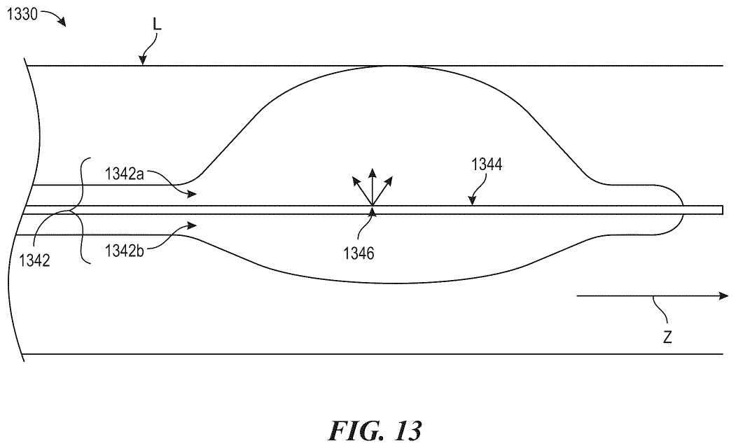

FIG. 13 is a side view of a distal portion of a cryotherapeutic device 1330 configured in accordance with some embodiments of the technology. The device 1330 is sized to fit with a lumen of an endoscope (e.g., the bronchoscope), and includes an expandable member 1342 (e.g., also referred to as “a balloon 1342”), an injection tube 1344 extending into the expandable member 1342, and at least one opening 1346 in the injection tube 1344 for emitting refrigerant into the expandable member 1342. If the endoscope has a camera that is non-concentric with respect to the device 1330, the balloon 1342 may be non-concentric with respect to the device 1330 but concentric with respect to the camera so that the camera can capture images that accurately reflect the surroundings of the balloon 1342 from the perspective of the balloon 1342. The balloon 1342 may be non-occlusive. As illustrated, the balloon 1342, when inflated, may include a first section 1342a that is configured to appose a portion of an internal wall of a body lumen L and a second section 1342b that is smaller than the first section 1342a and does not appose the internal wall of the body lumen L. Accordingly, the balloon 1342, when inflated, may achieve apposition and therefore facilitate effective heat transfer between the refrigerant and the tissue at or near where the balloon 1342 apposes the internal wall of the lumen L; meanwhile, the inflated balloon 1342 does not occlude the body lumen L to completely block fluid communication across the inflated balloon 1342 (along or against the direction of the axis Z). The injection tube 1344, and/or an ancillary lumen (e.g., the ancillary lumen 780 described above), may extend beyond the balloon 1342.

Non-occlusive expandable members, e.g., those as illustrated in FIGS. 8A-13, may allow a path for fluid communication between various portions of the body lumen (e.g., an airway) that are otherwise separated or blocked (e.g., by an inflated balloon of a catheter) so as to facilitate/achieve one or more of lung venting or mucus clearance. For example, the space not occluded by the inflated balloon (also referred to as non-occluded space) may allow a mucus clearing device to access mucus in a portion of the airway near where the balloon apposes the internal wall of the airway and/or to enhance the suction capacity of the mucus clearing device. The non-occluded space may provide a delivery channel to deliver cryoprotectant to a desired location (e.g., a bronchus surface) before the balloon of the catheter expands so that the cryoprotectant may act as a mask or landing pad for the balloon when it expands to its inflated configuration and apposes the internal wall of a body of the object.

The non-occluded space may allow position or passage of a guidewire or a measurement component. For example, the non-occluded space may allow position or passage of a guidewire configured to assist an endoscope (e.g., the bronchoscope 180) in delivery of a balloon of a catheter to challenging anatomy. As another example, the non-occluded space may allow position or passage of a measurement component (e.g., a flexible guidewire or pigtail) configured to measure the internal diameter (“ID”) of the body lumen (e.g., a bronchus). This may obviate the need to prescreen patients. As a further example, the non-occluded space may allow position or passage of an electrophysiology (EP) mapping catheter for EP mapping of bronchi so as to combine EP mapping of the bronchi and esophagus for confirming the relative position of the catheter before proceeding with the application of refrigerant to or near the target site. This may allow monitor proximity of a distal portion of the catheter (where the refrigerant application is effectuated) to the esophagus and ensure/improve procedural safety.

In some embodiments, a non-occlusive balloon may allow split denervation and avoid a 360-degree denervation in a cross-section that may impair normal functions of the treated area or areas that are nearby or downstream/distal to the treated area. A non-occlusive balloon may allow split cartilage freezing.

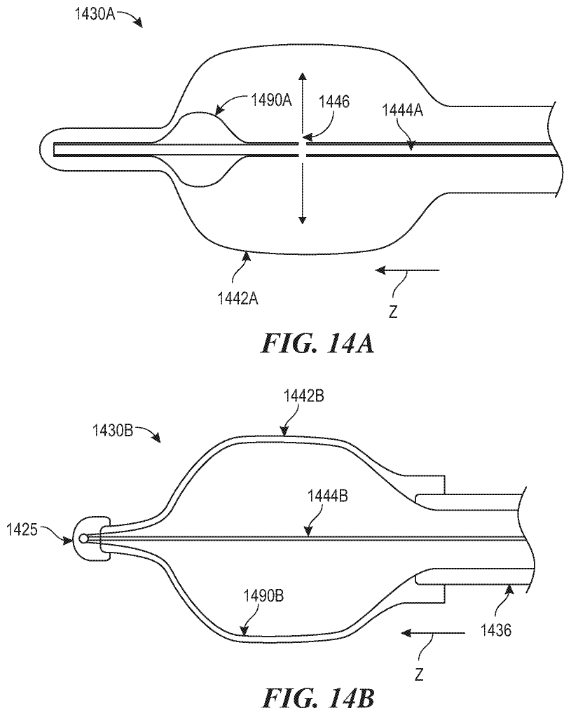

FIGS. 14A and 14B illustrate side views of a distal portion of cryotherapeutic devices 1430 (“device 1430”; referred to as a first device 1430A and a second device 1430B, respectively) having dual-balloon structures configured in accordance with some embodiments of the technology. The devices 1430 include various features similar to the cryotherapeutic devices described herein, including a delivery shaft 1436, a first balloon 1442 (also referred to as an outer balloon) securely and/or substantially hermetically coupled to the delivery shaft 1436, and an injection tube 1444. The device 1430 may further include a second balloon 1490 (also referred to as an inner balloon, an internal balloon, and the like) disposed within the first balloon 1442. The second balloon 1490 may serve to accurately position the outer balloon 1442 with respect to the injection tube 1444 (e.g., center the outer balloon 1442 around the injection tube 1444) and/or within the target lumen (e.g., center the outer balloon 1442 within the airway), and/or control the shape of the outer balloon 1442 for delivery and retraction into an endoscope (e.g., a bronchoscope). In some embodiments, the second balloon 1490 may be replaced by and/or used in conjunction with one or more expandable members having a different construction, such as an expandable cage or mesh structure (e.g., an expandable nitinol (NiTi) structure). Similar to the second balloon 1490, such an expandable structure can facilitate positioning of the outer balloon 1442 relative to other components of the device 1430 (e.g., the injection lumen 1444), support proper positioning of the outer balloon 1442 within the lumen at the target site, and/or at least partially define the exterior profile of the outer balloon 1442 when expanded.

As shown in FIG. 14A, the inner balloon 1490A can extend only along a portion of the outer balloon 1442A, e.g., along a distal section and/or along a proximal section) to support positioning (e.g., centering) of the injection tube 1444A within the first balloon 1442A. In other embodiments The inner balloon 1490A can be fluidly coupled to a separate inflation source (e.g., air) other than the refrigerant to provide for independent expansion of the inner balloon 1490A.

As shown in FIG. 14B, the inner balloon 1490B may extend along a substantial length of the outer balloon 1442B (e.g., along the entire length of the outer balloon 1442B, more than half of the length of the outer balloon 1442B). The outer balloon 1442B may, when inflated, appose an internal wall of a body lumen to facilitate effective heat transfer between the refrigerant and the tissue at or near where the outer balloon 1442B apposes the internal wall, thereby effectuating cryoablation of the tissue. Accordingly, the outer balloon 1442B may also be referred to as an ablation balloon. The injection tube 1444B may be substantially concentric with the internal balloon 1490B and/or the ablation balloon 1442, and/or extend through a substantial portion along the length of the internal balloon 1490B and/or the ablation balloon 1442B. As further shown in FIG. 14B, the device 1430B (and any other cryotherapeutic devices disclosed herein) may include a protector 1425 at its distal end to provide an atraumatic leading surface in the event the distal end comes into contact with the internal wall of the body lumen when the device 1430B is advanced through the body lumen.

In some embodiments, the injection tube 1444 is fluidly connected to the outer balloon 1442 by the openings 1446 and fluidly isolated from the inner balloon 1490. The inner balloon 1490 may be inflated (e.g., to position the outer balloon 1442) by a secondary lumen (e.g., a separate lumen extending through the injection tube 1444 or a tube separate from the injection tube 1444) that extends from a proximal portion of the catheter to a chamber of the inner balloon 1490. The inner balloon 1490 may be supplied by a fluid such as air. In other embodiments, the injection tube 1444 is fluidly coupled to both the inner balloon 1490 and the outer balloon 1442. For example, the injection tube 1444 may have a lumen that dispenses refrigerant or other fluid within the inner balloon 1490 and a separate lumen supplying refrigerant and/or other fluid to the outer balloon 1490. In some embodiments, the outer and inner balloons 1442, 1490 are fluidly couple to each other (e.g., via valves, openings). In embodiments where the inner balloon 1490 is replaced with a mesh or other expandable structure, the expandable structure may include one or more openings such that refrigerant is expelled into the outer balloon 1442. In further embodiments, the injection tube 1444 is fluidly connected to the inner balloon 1490 by the openings 1446 and fluidly isolated from the outer balloon 1442. In such embodiments, the refrigerant may be expelled into the inner balloon 1490 for effectuating cryoablation to the tissue near where the outer balloon 1442 apposes the internal wall. A space between the inner balloon 1490 and the outer balloon 1442 may be supplied by a secondary lumen. In some embodiments, the space may be filled by a thermally conductive fluid.

FIGS. 15A-15D illustrate various views of distal portions of cryotherapeutic devices configured to provide narrow thermal profiles on balloon surfaces in accordance with some embodiments of the technology. FIG. 15A illustrates a schematic side view of a distal portion of a cryotherapeutic device 1530A (“device 1530A”) including an expandable member 1542A (e.g., a balloon) around an injection tube 1544A. The injection tube 1544A may spray or otherwise emit refrigerant into the balloon 1542A via openings 1546A distributed (e.g., in a single row) around a section of the injection tube 1544A. When in the expanded configuration, a portion of the balloon 1542A may protrude outwardly to provide a narrow ablation region 1543 (also referred to as an ablation region, a bulging portion, and the like) configured to appose (e.g., press against) an internal wall of a body lumen. The ablation region 1543 of the balloon 1542A may substantially correspond to the section where the openings 1546A of the injection tube 1544A are located. For example, in the illustrated embodiment, the bulging, ablation region 1534 is positioned along a central portion of the expandable member 1542A (e.g., referred to as the equator of the balloon 1542A). The ablation region 1534 of the balloon 1542A and the section of the injection tube 1544A where the openings 1546A are distributed may be short (e.g., extend along a short longitudinal segment) so as to create a narrow thermal profile 1555 on the balloon surface and also in the tissue at or near where the balloon 1542A apposes the internal wall of the body lumen. In some embodiments, the expandable member 1542A may include multiple bulging portions spaced at a longitudinal distance from one another. This may allow for multiple narrow thermal profiles 1555 to be created simultaneously by the expandable member 1542A.

FIG. 15B is a side view of a distal portion of a cryotherapeutic device 1530B including a balloon 1542B around an injection tube 1544B. The injection tube 1544B may include a proximal section 1544-1 and a distal section 1544-3 that are substantially concentric with the balloon 1542B and an active section 1544-2 between the proximal and distal sections 1544-1 and 1544-3. The active section 1544-2 may have the shape of a loop and/or other shape that positions this section of the injection tube 1544 closer to a portion of the balloon 1542B that presses against an inner wall of a body lumen and therefore closer to the portion of the tissue of the internal wall. The active section 1544-2 may be a single loop or may have the shape of multiple loops. The active section 1544-2 may include one or more openings through which refrigerant may be emitted into the balloon 1542B. The one or more openings may be disposed on an outward facing surface of the active section 1544-2. The configuration of the injection tube 1544B may create a narrow thermal profile on the balloon surface and also along the tissue at or near where the balloon 1542B apposes the internal wall of the body lumen. In some embodiments, proximal and distal sections 1544-1 and 1544-3 include one or more openings that are further from the portion of the tissue of the internal wall. This may be such that a narrow thermal profile is created at an active portion 1544-2 while secondary thermal profiles are created at the proximal and distal sections 1544-1 and 1544-3.

FIG. 15C is a schematic side view of an injection tube 1544C for a cryotherapeutic device configured in accordance with some embodiments of the technology. The injection tube 1544C may have a single lumen in a proximal portion and split into multiple secondary lumens in a distal portion. Alternatively, the injection tube 1544C may include multiple secondary lumens that are bundled together in a proximal portion and unbundled along a distal portion. The multiple secondary lumens of the injection tube 1544C may have openings 1546C distributed circumferentially in a cross-section through which refrigerant is emitted into a balloon or other expandable member 1542C. The cross-section may be a short region along the length direction of a balloon 1542C so as to create a narrow thermal profile on the balloon surface. In addition, the opening 1546C may be positioned closer to the balloon surface to facilitate a targeted and/or focused refrigerant delivery. For example, the injection tube 1530C may be used with a balloon 1542C substantially similar to 1542A in which the cross-section where the openings 1546C are distributed may substantially corresponds to the equator of the balloon. As another example, the injection tube 1544C may be used with a balloon 1542C substantially similar to 1542B in which the cross-section where the openings 1544C are distributed may substantially corresponds to the equator of the balloon 1542B. The expandable member 1542 with the injection tube 1544C may be similar to any of the example described previously.

In some embodiments, the openings 1546C are located at distal ends of the multiple secondary lumens, along a length of the multiple secondary lumens, or a combination thereof. The multiple secondary lumens may extend to a distal end of the expandable member 1542C. In such embodiments, the multiple secondary lumens may join together at the distal end or be bundled together at the distal end. In other embodiments, the multiple secondary lumens extend partially through the expandable member 1542C. The multiple secondary lumens may terminate at an equator of the expandable member 1542C and/or another portion of the expandable member 1542C that aligns with a narrow thermal profile.

FIG. 15D illustrates a schematic of a narrow thermal profile 1565 that may be achieved by an expandable member 1542D in accordance with embodiments of the present technology. A narrow thermal profile may be achieved via an expandable member 1542D having a short equator region (e.g., similar to the balloon 1542A as illustrated in FIG. 15A), an injection tube 1544D with openings 1546D distributed over a short section and/or close to the balloon surface (e.g., the injection tube 1544D similar to 1544B as illustrated in FIG. 15B or the injection tube 1544C as illustrated in FIG. 15C), or the like, or a combination thereof. A narrow thermal profile may also be achieved by including thermal insulation outside a narrow working length of the balloon 1542D and/or an injection tube 1544D with openings 1546D at the equator of the balloon 1542D (e.g., the openings 1546A as illustrated in FIG. 15A). The thermal profile in the tissue may substantially correspond to where cryoablation occurs. A narrowed thermal profile at the balloon surface, which may substantially correspond to where cryoablation occurs, may focus cryoablation to a targeted and focus region while minimizing or reducing impact to surrounding tissue. The narrow thermal profile may diffuse farther from the balloon into the tissue, where the nerves are located. In other embodiments, thermal insulation may be applied to the balloon 1542D differently. For example, thermal insulation may be applied to a subsection of the balloon 1542D to prevent or reduce cryoablation of a patch of tissue. In some embodiments, thermal insulation may be applied at a proximal and distal end of the balloon 1542D. In some embodiments, thermal insulation may be applied such that two narrow thermal profiles are created. This may be such that cryoablation may be performed at either side of a cartilage ring simultaneously.

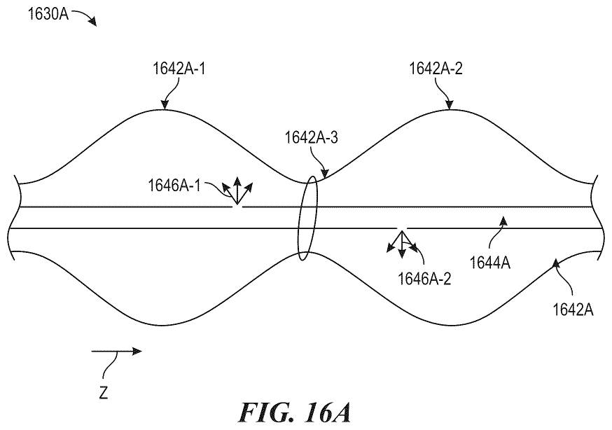

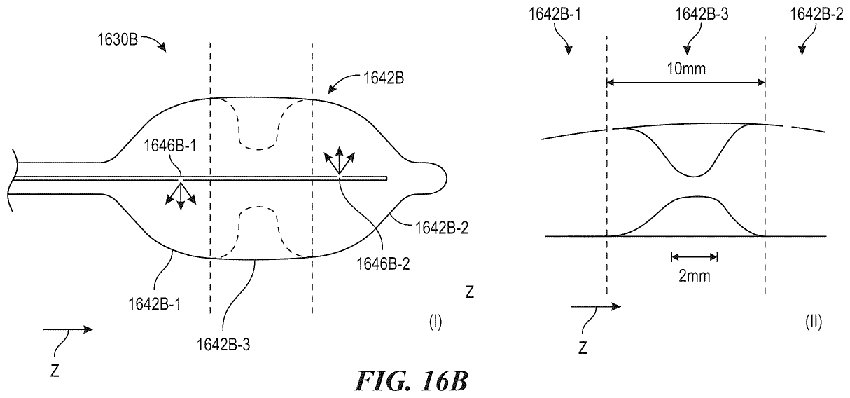

A body lumen, such as the trachea or bronchi in the respiratory system, may include cartilage rings to maintain the structural integrity, flexibility, and functionality (e.g., sound production). Accordingly, in a cryotherapy procedure performed through a body lumen including cartilage rings, cartilage preservation may need to be taken into consideration. Cartilage preservation, as well as an efficient cryoablation of a target site, may be achieved by modulating the thermal profile such that the target site is exposed to a sufficiently low temperature to induce cryoablation while a cartilage ring or multiple rings are protected from such a low temperature. Such a desired thermal profile may be achieved by configuring a balloon shape, a manner of refrigerant injection, or the like, or a combination thereof. Cartilage preservation may be achieved by a narrow or otherwise well-defined thermal profile so that the thermal profile effective for cryoablation directed to a target site has a low impact on a cartilage ring located in a vicinity of the target site. See, e.g., FIGS. 6G and 15A-15D, and relevant description thereof. Cartilage preservation may also be achieved by specific balloon shapes so that discontinuous apposition locations (between the balloon and an internal wall of a body lumen) corresponding to one or more target sites are created and a cartilage ring located in a vicinity of the one or more target sites are positioned at an interval between the discontinuous apposition locations. See, e.g., FIG. 11A 12C, and relevant disclosure thereof. Additional examples of catheter configurations for cartilage preservation in a cryotherapy procedure may be found in FIGS. 16A and 16B illustrating schematic views of distal portions of catheters in accordance with some embodiments of the technology.

FIG. 16A is a side view of a distal portion of a cryotherapeutic device 1630 (device 630) including a balloon 1642A around an injection tube 1644A. The balloon 1620A, when inflated, may include multiple equators (also referred to as lobes) including first and second equator sections 1620A-1 and 1642A-2 that appose an internal wall of a body lumen at two separate apposition locations and are connected by a narrow section 1642A-3 that is spaced from and not in contact with the internal wall. The narrow section 1642A-3 may correspond to where a cartilage ring is located underneath the internal wall of the body lumen. The injection tube 1644A may include cryoports (or cryoport clusters each including multiple cryoports) 1646A-1 and 1646A-2 positioned at locations that substantially correspond to the equators 1642A-1 and 1642A-2. The equator sections 1642A-1 and 1642A-2 of the balloon 1642 may substantially correspond to ablation sections of the balloon 1642A where heat transfer between refrigerant and the internal wall (and the underlying tissue) is sufficient to effectuate cryoablation. While heat transfer between the refrigerant in the balloon 1642A and the internal wall (and the underlying tissue) at the equators 1642A-1 and 1642A-2 is high, heat transfer between the refrigerant and the internal wall (and the cartilage ring) at the narrow section 1642A-3 is much lower, thereby preserving the cartilage ring.

The narrow section 1642A-3 may be formed in a number of different ways, some of which may be similar to creation of the indents described with reference to FIG. 10. The narrow section 1642A-3 may be defined by a ring of thicker material. In other embodiments, a ring may be applied to the narrow section 1642A-3 to constrain radial expansion of the expandable member 1642A. The ring may be made of a same or different material as the expandable member. The narrow section 1642A-3 may have an original diameter less than that of the equator sections when deflated.

Panel I of FIG. 16B illustrates a schematic side view of a distal portion of a cryotherapeutic device 1630B (“device 1630B”) including a balloon 1642B around an injection tube 1644B. The balloon 1642B, when inflated, may appose an internal wall of a body lumen over a continuous section that spans at least two neighboring cartilage rings. The injection tube 1644B may have two cryoports (or cryoport clusters) 1646B-1 and 1646B-2 positioned at locations separated from each other. Each of the two cryoports (or cryoport clusters) 1646B-1 and 1646B-2 may produce a narrow thermal profile on an ablation section (1642B-1 and 1642B-2, respectively) on the balloon 1642B where heat transfer between refrigerant in the balloon 1642B and the internal wall (and the underlying tissue) is sufficient to effectuate cryoablation. Conversely, heat transfer between refrigerant in the balloon 1642B and the internal wall (and the underlying tissue) at a middle section 1642B-3 between the ablation sections 1642B-1 and 1642B-2 may be low. The middle section 1642B-3 may substantially correspond to where a cartilage ring is located underneath the internal wall of the body lumen. Accordingly, the cartilage ring may be preserved in the procedure. As illustrated in panel II of FIG. 16B, the middle section 1642B-3 may be approximately 10 millimeters wide along the Z-axis, and the portion of the middle section 1642B-3 may be approximately 2 millimeters wide. The middle section 1642B-3 may also be coated with thermal insulation as described with reference to FIG. 15D to further decrease ablation.

As described above, the material of the balloon may be compliant or non-compliant so that the balloon may exhibit a desired shape to appose a specific area of the internal wall of the airway to achieve targeted lung denervation without significant damage to the surrounding healthy tissues. The material of the balloon may be substantially inert to the refrigerant, substantially insert to and biocompatible with the physiological environment of the airway, and/or sustain the temperature of the refrigerant at which cryotherapy proceeds. For example, the balloon may be made of polyurethane, polyethylene, polytetrafluoroethylene (PTFE), polyvinyl chloride (PVC), nylon, PEBAX, silicone, polyimide, polypropylene, or the like, or a combination thereof.

In addition to the shape, a balloon may be configured to exhibit other properties to facilitate the performance of a cryotherapy procedure and/or its efficacy. For example, a balloon may include a mesh texture on the outer surface that faces the body lumen to gather and use mucus for cryoprotection. The mesh may contain a desired component (e.g., dried elements that can be “activated” by moisture of mucus). As another example, a balloon may be made of a porous material that contain seats for a desired component. Such a desired component may include a gel coating including cryoprotectant gel. In some embodiments, the gel may also function as a lubricant to facilitate passage of the balloon through the lumen of the endoscope (e.g., the bronchoscope 160 as illustrated in FIG. 1) and/or the body lumen. As a further example, in combination with any one of various balloon shapes described elsewhere in the present document, a balloon may be configured with varying thickness along its length. Merely by way of example, the balloon may have a thicker layer corresponding to an injection area of the balloon (close to the cryoports on the injection tube through which refrigerant is sprayed into the balloon) for protecting certain tissue located at or near where the balloon apposes an internal wall of a body lumen (e.g., epithelial protection) from extreme low temperature (by impeding the heat transfer between the refrigerant and the tissue across the thicker layer).

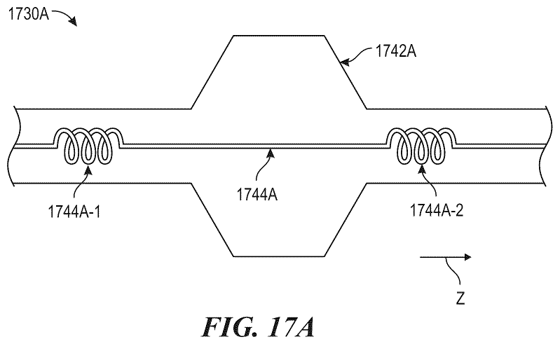

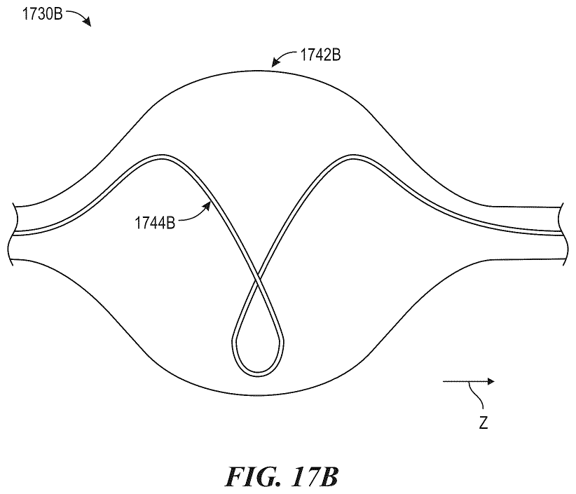

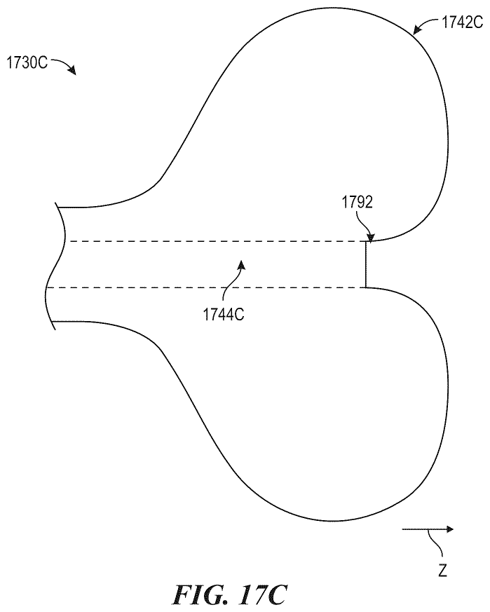

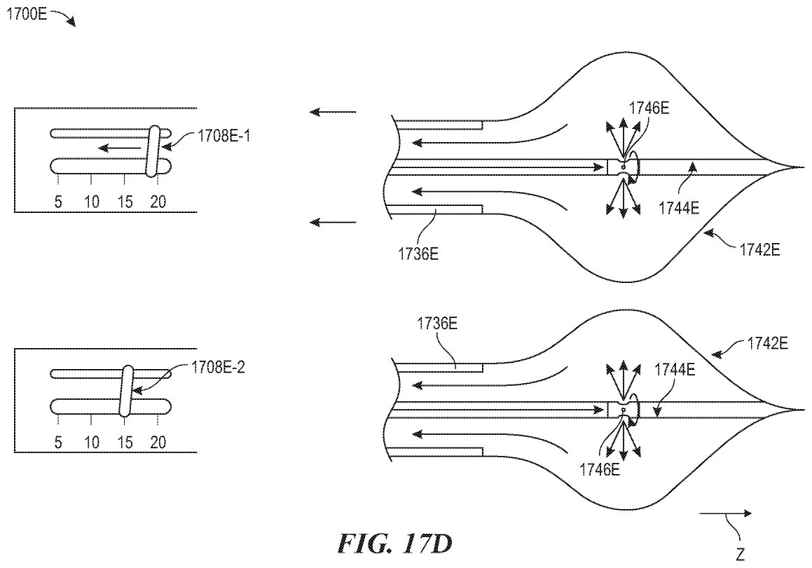

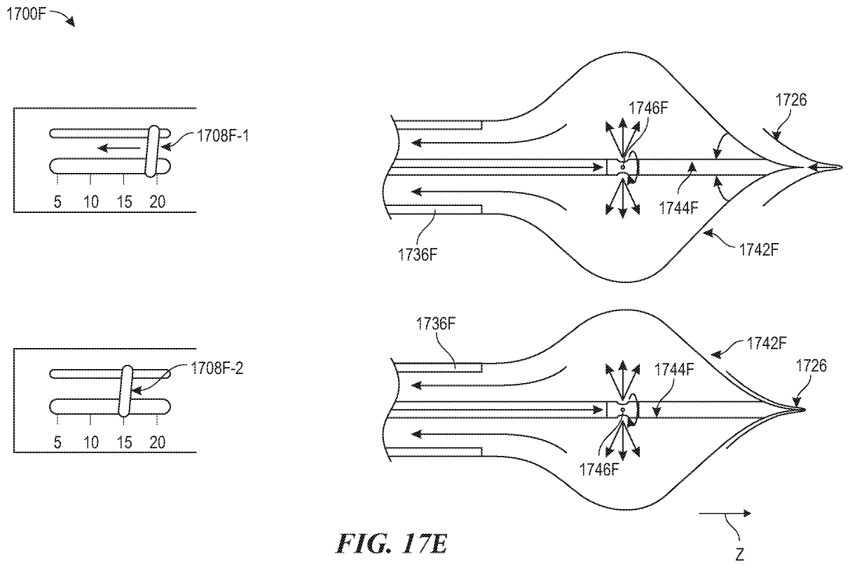

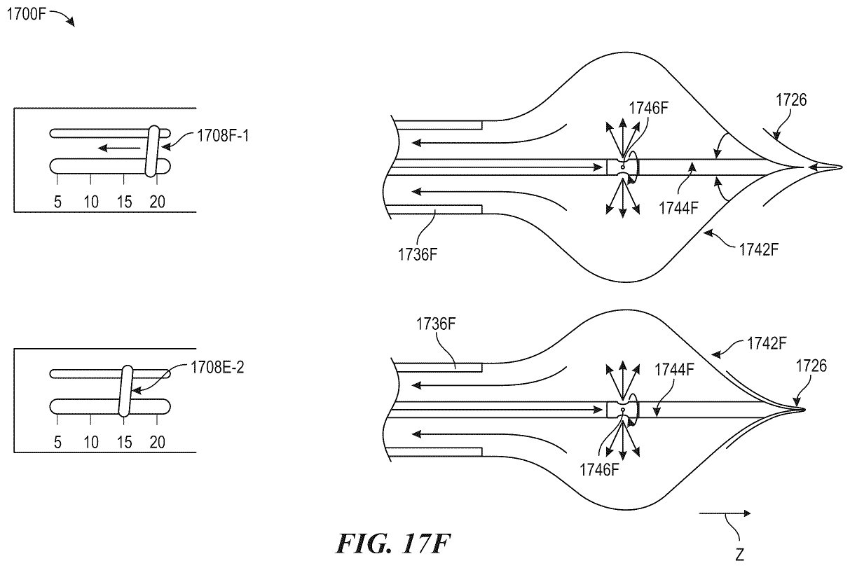

A catheter may be configured to allow for a wide range of adjustments to accommodate inter-patient variations in the anatomy including, e.g., the geometry of a body lumen where a balloon of the catheter is to be positioned to perform cryoablation. Examples of relevant anatomical features may include tortuosity, diameter, length, surrounding tissue or organs, or the like, or a combination thereof, of the body lumen. Such an adjustment may be achieved in vivo during a cryotherapy procedure so that a single catheter design may fit a large patient population, and also eliminate the need for prescreening patients. For example, the balloon shape and/or size may be adjusted after apposition. FIGS. 17A-17E illustrate examples of adjustable balloon configurations according to some embodiments of the technology.

FIG. 17A illustrates a distal end of a cryotherapeutic device 1730A (“device 1730A”) including a balloon 1742A around an injection tube 1744A configured in accordance with the present technology. The injection tube 1744A may include one or more spring sections (also referred to as a spiral section or a resilient section) 1744A-1 and 1744A-2, on either end of the working length of the balloon 1742A to ensure cryoports are centered within the balloon 1742A. In some embodiments, the injection tube 1744A may be independent of the spring so that the properties of refrigerant in the injection tube 1744A do not change when the spring compresses or extends. In some embodiments, the spring section may be configured to allow an adjustment of the cryoports to bias their positions within the balloon 1742A. The adjustment of the cryoports within the balloon 1742A may be achieved by other mechanisms described elsewhere in the present documents (e.g., the sheath as illustrated in FIG. 6E). For example, the length of the spring sections 1744A-1 and 1744A-2 may be controlled by an actuator operably connected to the injection tube 1744A. The actuator may be controlled at a handle of the device 1730A (e.g. similar to handle 138 of device 130).

FIG. 17B illustrates a distal end of a cryotherapeutic device 1730B (“device 1730B”) including a balloon 1742B around an injection tube 1744B configured in accordance with the present technology. The injection tube 1744B may have the shape of a spiral, a loop, and/or a helical section. The shaped section may stretch or retract to position the cryoports in a central or otherwise desired position within the balloon 1742B, or to allow an adjustment of the cryoports to bias their positions within the balloon 1742B. In some embodiments, one or more openings located on the injection tube 1744B may located on an outer surface such that a refrigerant may be expelled closer to the tissue of an internal surface of the lumen.

FIG. 17C is a side view of a distal end of a cryotherapeutic device 1730C (“device 1730C”) including a balloon 1742C around an injection tube 1744C configured in accordance with the present technology. A distal tip 1792 of the balloon 1742C may be coupled to a device configured to advance or retract the distal tip 1792. In some embodiments, the distal tip may be retracted during delivery of the device 1730C into the lumen and then advanced once placed for ablation (e.g., as described above with respect to FIG. 4). In some embodiments, the balloon 1742C may be coupled to a device configured to advance or retract a proximal tip of the balloon 1742. The device may be a floating shaft that is concentric to the balloon 1742C. In some embodiments, the balloon 1742C may be inverted utilizing an internal feature of the balloon 1742 to change the diameter (e.g., increase or decrease) of the balloon 1742C. In some embodiments, the distal tip 1792 is operably connected to the injection tube 1744C and the injection tube 1744C may be controlled to advance or retract the distal tip 1792.

FIG. 17D illustrates schematic views of a distal portion of a cryotherapeutic device 1730E (“device 1730E”) including a balloon 1742E and an injection tube 1744E configured in accordance with the present technology. The device 1730E may include a shaft 1736E (e.g., a delivery shaft, an exhaust shaft), the balloon 1742E securely and/or substantially hermetically coupled to the shaft 1736E, and the injection tube 1744E. The injection tube 1744E may include one or more cryoports 1746E. In some embodiments, the injection tube 1744E also includes a rotary device that rotates to dispel refrigerant within the balloon 1742E (e.g., similar to that described with reference to FIG. 6I above). The balloon 1742E may be pulled, rotated, or otherwise moved (via an actuator at the proximal end) along or against the direction of the Z-axis (as illustrated by the 1708E-1 vs. 1708E-2) to change the shape of the balloon 1742E. An actuator 1708E (also referred to as a controller; e.g., positioned along a proximal handle operably coupled to the distal portion of the device 1730E) may be moved or otherwise actuated to change a distance the distal end moves and/or how much the shape of the balloon 1742E is changed. After moving the actuator 1708E from a first position 1708E-1 to a second position 1708E-2, the distal end of the balloon 1742E is moved and the size of the balloon 1742E (e.g. the expanded diameter) changes (e.g., increases or decreases depending upon the manner of connection). The actuator 1708E may be operably connected to a separate controller which is configured to move (e.g., pull) the proximal end and/or distal end of the balloon 1742E to change its shape.

FIG. 17E illustrates schematic views of a distal portion of a cryotherapeutic device 1730F (“device 1730F”) including a balloon 1742F around an injection tube 1744F configured in accordance with the present technology. The device 1730F may include a shaft 1736F (e.g., a delivery shaft, an exhaust shaft), a balloon 1742F securely and/or substantially hermetically coupled to the shaft 1736F, and an injection tube 1744F. The injection tube 1744F may include one or more cryoports 1746F. Similar to the balloon 1742E, the balloon 1742F may be pulled at the proximal end along or against the direction of the Z-axis (as illustrated by the 1708E-1 vs. 1708E-2) to change the shape of the balloon 1742F. In addition, the balloon 1742F may also be pulled at a distal end 1726 along or against the direction of the Z-axis. In some embodiments, pulling a distal end 1726 proximally increases an expanded diameter of the balloon 1742 and, in some embodiments, decreases a chamber volume of the balloon 1742. The mechanism to control this change may be substantially similar to that described with reference to FIG. 17D. Moving an actuator 1708F from a first position 1708F-1 to a second position 1708F-2 may pull or otherwise move the distal end 1726.

The cryotherapeutic devices may include an exhaust lumen configured to remove refrigerant exhaust away from a body lumen to an exhaust reservoir. The sidewall of the delivery shaft of a catheter may serve as a sidewall of the exhaust shaft. In some embodiments, the exhaust shaft may have its own sidewall other than the sidewall of the delivery shaft. For illustration purposes and for brevity, the following description is provided with respect to the exhaust shaft. It is understood that the exhaust shaft may be the same structure as or a separate structure different from the delivery shaft described elsewhere in the present document.

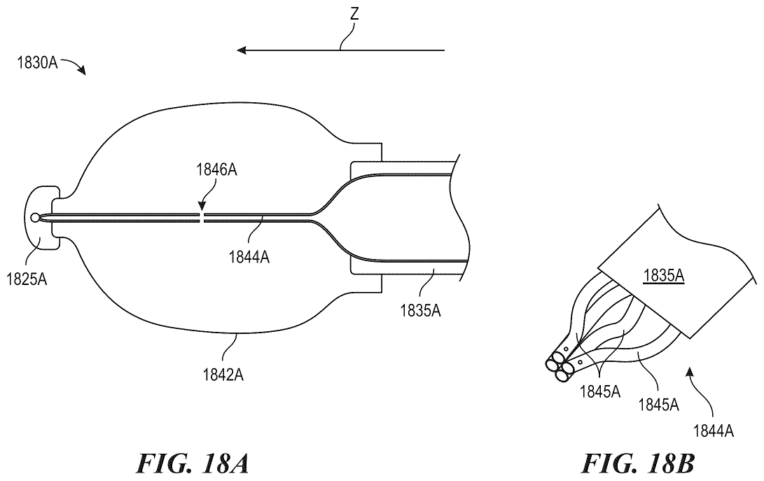

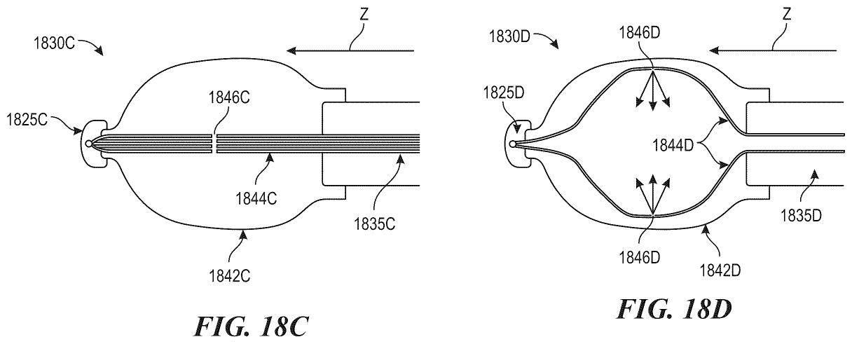

The exhaust shaft may be configured to have sufficient pushability, stiffness, and/or flexibility to be pushed through the lumen of an endoscope and/or through a body lumen. The exhaust shaft may also provide support and/or protection for the delivery line, the injection tube, the balloon, or the like, or a combination thereof, of the catheter so that one or more of these components, as well as refrigerant, may be delivered to or away from a target site through a body lumen. The exhaust shaft may also facilitate cycle stability by better maintaining the temperature of refrigerant when it is delivered through the delivery line from a refrigerant source (e.g., a cryoconsole) to a target site in a body lumen of an object. In addition, the exhaust shaft may need to have a low profile to fit in a lumen of an endoscope (e.g., a bronchoscope). FIGS. 18A-18D illustrate examples of configurations of exhaust shafts according to some embodiments of the technology.

FIG. 18A illustrates a schematic side view and a schematic perspective view of a distal portion of a cryotherapeutic device 1830A (“device 1830A”) configured in accordance with embodiments of the technology, and FIG. 18B is an enlarged view of an injection tube configuration of the device 1830A. Referring to FIG. 18A, the device 1830A may include an exhaust shaft 1835A, a balloon 1842A securely and/or substantially hermetically coupled to the exhaust shaft 1835A, and an injection tube assembly 1844A including one or more cryoports 1846A. The one or more cryoports 1846A may be located on an outward surface of the bundle of injection tubes 1845A. The device 1830A may include a protector 1825A at its distal end (e.g., at a distal portion of the balloon 1842A or the injection tube assembly 1844A). The protector 1825A may help avoid the tip of the catheter 1830A does not injure the internal wall of the body lumen or the balloon 1842A.

As shown in FIG. 18B, the injection tube assembly 1844A may may include a bundle of injection tubes 1845A (also referred to as secondary tubes) that each deliver refrigerant to the interior of the balloon 1842A (FIG. 18A). The individual injection tubes 1845A may be distributed around the circumference of the exhaust shaft 1835A (e.g., around the internal wall of the exhaust shaft 1835A), thereby adding crush resistance to the smaller diameter injection tubes 1845A and pushability to the exhaust shaft 1835A. This configuration is also expected to help maintain refrigerant temperature within the individual injection tubes 1845A (e.g., avoid freezing) due to the individual injection tubes 1845A direct proximity to the exhaust lumen, which is warmed by the temperature external to the exhaust lumen (e.g., temperature in the airways, body heat). In some embodiments, the individual injection tubes are distribute throughout the exhaust shaft 1835A. The portion of the injection tube assembly 1844A extending beyond the exhaust shaft 1835A may be positioned concentrically in a central region of the balloon 1842A, offset within the balloon 1842A, and/or otherwise oriented within the balloon 1842A. In some embodiments, the bundle of injection tubes 1845A may converge at a distal end of the exhaust shaft 1835A to be more closely positioned within the balloon 1842A. In other embodiments, the individual injection tubes 1845A may advance proximally into the balloon with the same distribution as from inside the exhaust shaft 1835A. In further embodiments, the bundle of injection tubes 1845A may converge into an injection tube 1844A within the balloon 1842A. This injection tube may be substantially similar to any of the embodiments of injection tubes described previously.

FIGS. 18C and 18D illustrates a schematic side view of a distal portion of a cryotherapeutic device 1830C (“device 1830C”) configured in accordance with embodiments of the technology. The device 1830C may include an exhaust shaft 1835C, a balloon 1842C securely and/or substantially hermetically coupled to the exhaust shaft 1835C, an injection tube assembly 1844C including one or more cryoports 1846A, and a protector 1825C at the distal end of device 1830C (e.g., the balloon 1842C, or the injection tube assembly 1844C). The injection tube assembly 1844C may include a bundle of secondary tubes (e.g., similar to the configuration shown in FIG. 18B) that are centered or otherwise positioned along a portion of the length of the catheter (to achieve a minimal injection tube diameter for console performance) for delivery within the body lumen (e.g., to or away from a target site). A single injection tube of a first diameter may be less stiff than a bundle of secondary injection tubes, each having a diameter smaller than the first diameter, but whose overall bundle diameter is comparable to the first diameter (and thus delivering refrigerant at the same/similar volumetric rate as a single, larger diameter tube). The multiple secondary tubes of the injection tube assembly 1844C may allow the console or system to run closer to an optimal pressure.

In some embodiments, the bundle secondary tubes may run centric to the balloon 1842 as shown in FIG. 18C. The bundle of secondary tubes may be operably connected to one another with ribs, additional material, adhesive, rings, etc. In other embodiments, the bundle of secondary tubes may be shaped substantially to the balloon diameter as seen in FIG. 18D. The bundle of secondary tubes may be configured to expand outward as the balloon 1842D expands. The cryoports 1846D may be oriented inwards to allow for sufficient gas expansion and cooling. The cryoports 1846D may be oriented outwards such that they are closer to an internal wall of the lumen. The secondary tubes may expand to fit a variety of patient anatomies and a variety of target sites.

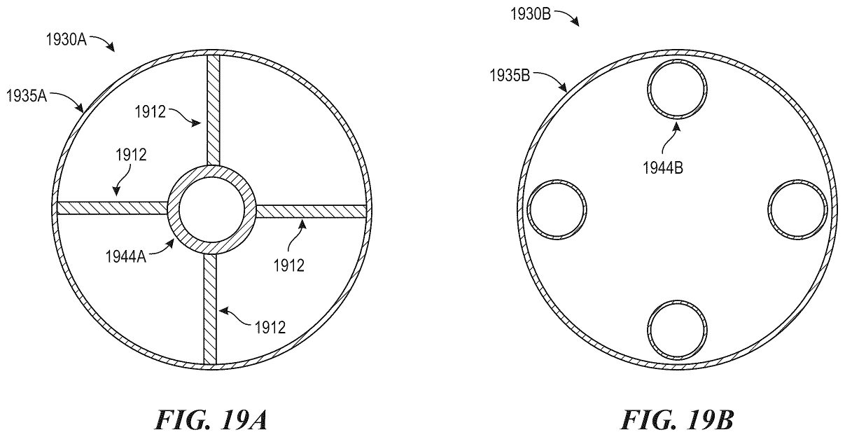

FIGS. 19A and 19B illustrate schematic cross-sectional views of cryotherapeutic devices configured in accordance with embodiments of the technology. FIG. 19A illustrates a device 1930A including an exhaust shaft 1935A and an injection tube 1944A located therein. The injection tube 1944A and the exhaust shaft 1935A may be configured as a single extrusion for simplified manufacturing. The exhaust shaft 1935A may concentrically enclose the injection tube 1944A with ribs 1912 between the injection tube 1944A and the exhaust shaft 1935A to maintain concentricity and structural stability. The injection tube 1944A may be composed of a material that thermally insulates the refrigerant substantially for consistent temperature delivery and to reduce (e.g., minimize) any blockages within injection lumen due to ice formation. In some embodiments, the injection tube 1944A includes a bundle of secondary tubes. In such embodiments, ribs 1912 may enclose the secondary tubes. The at secondary tubes may be disposed such that they may touch or with a spacing in between.

FIG. 19B illustrates a device 1930B including an exhaust shaft 1935B and an injection tube 1944B located therein. The injection tube 1944B may include a bundle of secondary tubes that are circumferentially distributed along the inner wall of the exhaust shaft 1935B. In some embodiments, the bundle of secondary tubes covers an entire circumference of the inner wall of the exhaust shaft 1935B. In other embodiments, there may be a spacing between at least two of the secondary tubes. The bundle of secondary tubes may be adhered to the inner wall. In other embodiments, the exhaust shaft 1935B and the injection tube 1944B are configured as a single extrusion for simplified manufacturing. The multiple injection lumens may maintain refrigerant temperature consistency, aided by the temperature external to the exhaust lumen. The multiple secondary injection tubes may increase cooling power while meeting pressure requirements of the console. The attachment of the secondary tubes onto the inner wall of the exhaust shaft 1935B may improve the pushability to the exhaust shaft and provide support to the secondary tubes.

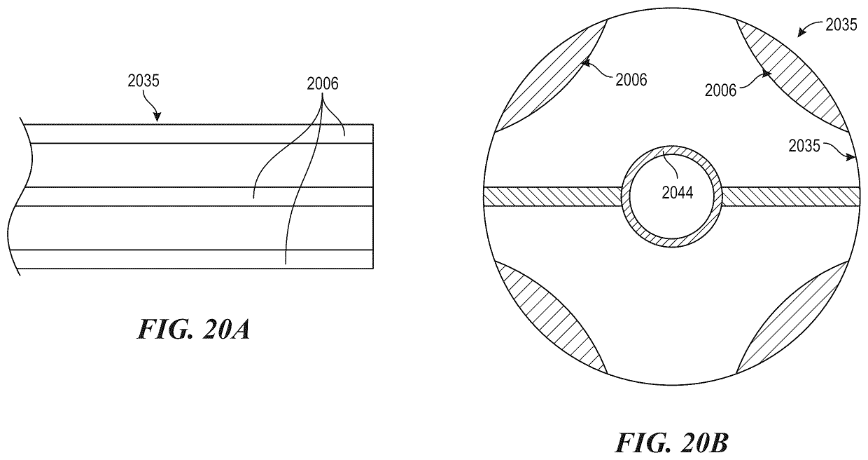

FIGS. 20A and 20B illustrate a schematic side view and cross-sectional view of cryotherapeutic devices including an exhaust shaft 2035 and an injection tube 2044 configured in accordance with embodiments of the technology. The injection tube 2044 may be disposed concentrically with the exhaust shaft 2035 and supported by multiple ribs. The exhaust shaft 2035 may contain channels for longitudinal flattened support wires 2006 or may include longitudinal flattened support wires 2006 distributed circumferentially along the inner wall of the exhaust shaft 2035. Substantially flattened support wires 2006 may provide a low profile, flexibility, kink resistance, exhaust patency, pushability to the exhaust shaft 2035. In some embodiments, the support wires 2006 may not extend into the distal end of the exhaust shaft 2035, thereby leaving the distal end free for flexibility and bonding to other components (e.g., a balloon). The support wires 2006 integrated into the exhaust shaft 2035 may provide additional steering of exhaust shaft 2035 from the proximal end beyond what is achieved by the endoscope (e.g., the bronchoscope 160 of FIG. 1A). In some embodiments, the flattened support wires 2006 may be made of a metal, plastic, or other semi-compliant material. The flattened support wires 2006 may be made of the material of the exhaust shaft.

Refrigerant exhaust may be delivered from the body lumen to an exhaust reservoir via the exhaust shaft. Liquid refrigerant (e.g., liquid nitrogen) may be delivered from a refrigerant source to the body lumen via a delivery line and/or an injection tube, whin may also be housed within the exhaust shaft. In view of the low temperatures of the liquid refrigerant and the refrigerant exhaust, the exhaust shaft may be actively heated by incorporating metallic elements within, e.g., exhaust braids so as to maintain the temperature of the exhaust shaft, increase cycle stability, and/or reduce ice formation on the exhaust shaft.

Various features of different components described herein may be applied alone or combined. For example, a specific balloon shape may be combined with a specific arrangement of the cryoports to achieve a desired performance. One or more other features may also be employed. For example, the catheter may include fixation or fixed positioning within the lumen of an endoscope (e.g., the bronchoscope 160) to lock a balloon position relative to the endoscope during ablation. As another example, the catheter housing or proximal portion (e.g., the strain relief, delivery shaft, the exhaust shaft) may be mechanically interlocked with the endoscope to achieve position stability during a cryotherapy procedure. The attachment of the catheter to the endoscope may provide one or more technical benefits including releasing strain or loop of the catheter into bronchoscope. Anchoring a balloon protector on the shaft may increase the straightened length to prevent kinking; in addition, the balloon protector may cover balloon during packaging and introduction into the endoscope. Further, at the proximal end of the balloon protector there may be a flare, to allow the balloon protector to slide over a strain relief to provide a more stable positioning of the balloon protector and reduce loss of working length of the catheter. In some embodiments, the system may be configured to provide two different mechanisms, a stopper (e.g., located proximal to the balloon) and feedback (intraprocedural signals, shaft marker) to the endoscope to remind a user not to push the catheter too far into the body lumen, and also provide grip in the case of the stopper. As a further example, the shaft may be configured to improve or maintain visibility during a procedure. For example, the shaft may include sponge or squeegee on an external side of the sidewall that faces the body lumen to absorb mucus or clean lens. As still a further example, the shaft may include clips configured to allow for stably positioning the catheter within the lumen of the endoscope. Interference between the exhaust shaft and Endobronchial Ultrasound seal (EBUS) may help maintain catheter positioning but also allow for repositioning of the distal end of the catheter (e.g., the balloon of the catheter).

### EXAMPLES

The following examples are illustrative of several embodiments of the present technology.
1. A cryotherapeutic system, comprising:
   an elongated shaft having a proximal end portion and a distal end portion configured to be delivered to a target site within a bronchial tree beyond the left or right main bronchus, wherein the shaft comprises a delivery line and an exhaust shaft;
   a handle disposed at a proximal end of the elongated shaft; and
   a cooling assembly at the distal end portion of the elongated shaft, the cooling assembly comprising—
      an expandable member having a flexible wall that defines a chamber, wherein the chamber is fluidically connected to the exhaust shaft, and wherein the expandable member has an expanded state in which the expandable member has an outer diameter of at least configured to contact an inner wall of an airway at the target site,
      an injection tube extending from the elongated shaft into the chamber, fluidically connected to the delivery line,
         wherein the injection tube comprises a plurality of openings configured to dispel refrigerant into the chamber such that the refrigerant expands to impart cryogenic thermal injury to neural fibers positioned a distance away from the inner wall of the airway.
2. The cryotherapeutic system of example 1 wherein the expandable member comprises a balloon made of a semi-compliant material.
3. The cryotherapeutic system of any one of the preceding or following examples wherein the expandable member has an outer diameter of at least 9 mm in the expanded state.
4. The cryotherapeutic system of any one of the preceding or following examples wherein the plurality of openings are arranged circumferentially around the injection tube in one or more rows.
5. The cryotherapeutic system of example 4 wherein the plurality of openings are arranged in at least two rows around the injection tube, and wherein individual openings in adjacent rows are offset from each other.
6. The cryotherapeutic system of any one of the preceding or following examples wherein at least one of the plurality of openings is wedge-shaped and tapers from an inner surface of the injection tube to an outer surface of the injection tube such that a dimension of the opening at the inner surface is larger than a dimension of the opening at the outer surface.
7. The cryotherapeutic system of any one of the preceding or following examples wherein the injection tube comprises a first portion having a first inner diameter and a second portion having a second inner diameter smaller than the first inner diameter, and wherein the plurality of openings are located at or near the second portion.
8. The cryotherapeutic system of any one of the preceding or following examples, further comprising a console operably coupled to the elongated shaft, the console comprising a supply component configured to retain a refrigerant at a pressure sufficient to maintain the refrigerant in a substantially liquid state at ambient temperatures.
9. The cryotherapeutic system of example 8 wherein the refrigerant comprises at least one of liquid nitrogen, nitrous oxide, carbon dioxide, or a hydrofluorocarbon.
10. The cryotherapeutic system of example 8 wherein the console further comprises a control component configured to control delivery of the refrigerant to the cooling assembly.
11. The cryotherapeutic system of any one of the preceding or following examples wherein the cooling assembly further comprises an atraumatic tip portion at a distal end of the expandable member.
12. The cryotherapeutic system of any one of the preceding or following examples wherein the cooling assembly further comprises one or more markers positioned along the injection tube and/or the distal end portion of the elongated shaft.
13. The cryotherapeutic system of any one of the preceding or following examples wherein the cooling assembly further comprises at least one sensor configured to detect at least one of temperature within the expandable member, pressure within the expandable member, and/or flow rate of the refrigerant.
14. The cryotherapeutic system of any one of the preceding or following examples further comprising a protector sheath component configured to slidably extend over at least a portion of the cooling assembly.
15. The cryotherapeutic system of example 14 wherein the protector sheath has a flared proximal region defining an opening larger in cross-sectional dimension than a distal region of the balloon protector component.
16. The cryotherapeutic system of example 14, wherein the protector sheath has a distal end region has an outer cross-sectional dimension of at least 2 mm such that at least a portion of the protector sheath is positioned outside a working channel of a bronchoscope when the cooling assembly is positioned within the working channel.
17. The cryotherapeutic system of any one of the preceding or following examples wherein the elongated shaft is sized to be delivered through a working channel of a bronchoscope.
18. The cryotherapeutic system of any one of the preceding or following examples wherein the expandable member is configured to transition between a delivery state having a low profile and the expanded state, and wherein in the expanded state the expandable member is configured to adhere to tissue at the target site upon delivery of the refrigerant.
19. The cryotherapeutic system of any one of the preceding or following examples wherein the cryogenic thermal injury is imparted to neural fibers positioned between 1 mm and 3 mm from the inner wall of the airway.
20. The cryotherapeutic system of any one of the preceding or following examples wherein the handle comprises attachment mechanisms configured to connect the handle to a handle of a bronchoscope.
21. The cryotherapy system of any one of the preceding or following examples wherein the injection tube further comprises a sheath partially surrounding an inner tube, wherein the plurality of openings are located along a length of the inner tube, and wherein the sheath is configured to move with respect to the inner tube to selectively place one or more of the plurality of openings in fluid communication with the chamber.
22. The cryotherapy system of any one of the preceding or following examples wherein the injection tube has a tube distal end with an outlet and a collar extending over the outlet, wherein the plurality of openings are located on the collar, and wherein the collar is movable with respect to the injection tube.
23. The cryotherapy system of any one of the preceding or following examples wherein the cooling assembly further comprises a rotary device operably coupled to the injection tube, wherein the rotary device is configured to rotate a location of the plurality of openings with respect to the expandable member.
24. The cryotherapy system of example 23 wherein the rotary device comprises a rotary turbine manifold configured to be driven by refrigerant moving through the rotary device.
25. The cryotherapy system of any one of the preceding or following examples wherein the cooling assembly further comprises an ancillary lumen extending at least from a proximal end portion of the expandable member to the distal end portion of the expandable member, and wherein the ancillary lumen is not fluidically coupled to the delivery line.
26. The cryotherapy system of example 25 wherein the ancillary lumen defines a bypass tube with a distal outlet positioned outside of the chamber and open to an external environment.
27. The cryotherapy system of any one of the preceding or following examples wherein the expandable member comprises at least one recessed region to define a plurality of lobes, wherein, when the expandable member is deployed at the target site, the recessed region is configured to define an open channel that allows airflow between a proximal end of the cooling assembly and a distal end of the cooling assembly.
28. The cryotherapy system of any one of the preceding or following examples wherein the expandable member comprises a first lobe and a second lobe positioned at least partially distal to the first lobe along a longitudinal axis of the cooling assembly, and wherein, when the expandable member is in the expanded stat, the first lobe projects in a first radial direction and the second lobe projects in a second radial direction different from the first radial direction.
29. The cryotherapy system of any one of the preceding or following examples wherein the injection tube comprises a plurality of individual injection tubes disposed about the exhaust shaft.
30. The cryotherapy system of any one of the preceding or following examples wherein the injection tube comprises a plurality of individual injection tubes bundled together along at least a portion of the cooling assembly.
31. The cryotherapy system of example 30 wherein the plurality of individual injection tubes are configured to extend outwardly from a longitudinal axis of the expandable member when the expandable member is in the expanded state such that the individual injection tubes have outlets positioned proximate an interior surface of the expandable member.
32. The cryotherapy system of any one of the preceding or following examples wherein the injection tube comprises a proximal section, a distal section, and an active section between the proximal and distal sections, and wherein the plurality of openings are positioned along the active section.
33. The cryotherapy system of any one of the preceding or following examples wherein, when in the expanded state, the expandable member has a protruding section that defines a narrow ablation region for contacting adjacent tissue.
34. The cryotherapy system of any one of the preceding or following examples wherein the expandable member has a thermal insulation positioned along at least a portion of the expandable member.
35. The cryotherapy system of any one of the preceding or following examples wherein the injection tube comprises at least one spring section configured to position the plurality of openings along a longitudinal axis of the cooling assembly that is at least partially aligned with an ablation region of the expandable member.
36. The cryotherapy system of any one of the preceding or following examples wherein the injection tube has a curved section configured to stretch or retract to position the plurality of openings in a central portion of the expandable member.
37. The cryotherapy system of any one of the preceding or following examples wherein the expandable member is coupled to an actuator at the proximal portion of the elongated shaft, the actuator configured to move the expandable member in a longitudinal direction to change a shape of the expandable member.
38. The cryotherapy system of any one of the preceding or following examples wherein the exhaust shaft comprises a plurality of support wires configured to provide patency of the exhaust shaft.
39. The cryotherapy system of any one of the preceding or following examples, further comprising at least one insulating element along the elongated shaft and/or the handle, and wherein the insulating element is configured to maintain a temperature within the exhaust shaft that avoids unregulated expansion of refrigerant flowing through the exhaust shaft.
40. The cryotherapy system of any one of the preceding or following examples, further comprising at least one heating element along the elongated shaft and/or the handle, and wherein the heating element is configured to maintain a temperature within the exhaust shaft that avoids unregulated expansion of refrigerant flowing through the exhaust shaft.
41. The cryotherapy system of any one of the preceding or following examples wherein, when the expandable member is deployed, the expandable member is configured to press against and conform to the inner wall of the airway, and wherein the expandable member is configured to treat airways having an inner diameter ranging from at least 3 mm and 10 mm. to have an outer diameter in the expanded state.
42. The cryotherapy system of any one of the preceding or following examples, further comprising a delivery channel with an outlet port open to an external environment and configured to dispense a cryoprotectant on an inner surface of the airway proximate to the target site.
43. A method of performing targeted lung neuromodulation, the method comprising:
   advancing a distal end portion of a cryotherapeutic device through a working channel of a bronchoscope to a target site beyond a left or right main bronchus;
   deploying an expandable member of a cooling assembly at the distal end portion at the target site such that the expandable member contacts and confirms to tissue along an inner wall of an airway;
   delivering refrigerant through an injection tube into the expandable member, wherein the refrigerant expands within the expandable member to provide cryotherapeutic cooling; and
   imparting cryogenic thermal injury to neural fibers positioned a distance away from the inner wall of the airway via the cryotherapeutic cooling.
44. The method of any one of the preceding or following examples wherein delivering the distal end portion to the target site comprises delivery the distal end portion to at least one of a right upper lobe bronchus, a right intermediate bronchus, a left upper lobe bronchus, and/or a left lower lobar bronchus.
45. The method of any one of the preceding or following examples, further comprising inflating the expandable member prior to delivering the refrigerant to confirm apposition of the expandable member against the inner wall of the airway.
46. The method of example 45, further comprising deflating the expandable member and repositioning the expandable member if adequate apposition is not achieved.
47. The method of any one of the preceding or following examples, wherein delivering the refrigerant causes ice to form around an exterior of the expandable member, thereby adhering the expandable member to surrounding tissue at the target site.
48. The method of any one of the preceding or following examples wherein imparting cryogenic thermal injury comprises imparting cryogenic thermal injury to the neural fibers positioned between 1 mm and 3 mm from the inner wall of the airway.
49. The method of any one of the preceding or following examples, further comprising directly visualizing the target site via the bronchoscope before, during, or after delivering the refrigerant.
50. The method of any one of the preceding or following examples wherein the cryotherapeutic cooling is delivered at a temperature of between −15 °C and −80 °C
51. The method of any one of the preceding or following examples, further comprising performing multiple ablation cycles of delivering refrigerant that imparts cryogenic thermal injury at the target site by deflating and reinflating the expandable member between cycles.
52. The method of any one of the preceding or following examples, further comprising heating a portion of an exhaust shaft as refrigerant moves through the exhaust shaft.
53. The method of any one of the preceding or following examples, further comprising applying a cryoprotectant to a portion of the inner wall of the airway before delivering the refrigerant.
54. The method of any one of the preceding or following examples, further comprising moving the cooling assembly through an introducer sheath at an inlet of the bronchoscope.

### conclusion

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

While particular embodiments of the present technology have been shown and described, it will be obvious to those skilled in the art that, based upon the teachings herein, changes and modifications may be made without departing from this technology and its broader aspects and, therefore, the appended claims are to encompass within their scope all such changes and modifications as are within the true spirit and scope of this technology. Furthermore, it is to be understood that the technology is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to technologies containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Conjunctive language, such as phrases of the form “at least one of A, B, and C,” or “at least one of A, B and C,” (i.e., the same phrase with or without the Oxford comma) unless specifically stated otherwise or otherwise clearly contradicted by context, is otherwise understood with the context as used in general to present that an item, term, etc., may be either A or B or C, any nonempty subset of the set of A and B and C, or any set not contradicted by context or otherwise excluded that contains at least one A, at least one B, or at least one C. For example, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}, and, if not contradicted explicitly or by context, any set having {A}, {B}, and/or {C} as a subset (e.g., sets with multiple “A”). Thus, such conjunctive language is not generally intended to imply that certain embodiments require at least one of A, at least one of B, and at least one of C each to be present. Similarly, phrases such as “at least one of A, B, or C” and “at least one of A, B or C” refer to the same as “at least one of A, B, and C” and “at least one of A, B and C” refer to any of the following sets: {A}, {B}, {C}, {A, B}, {A, C}, {B, C}, {A, B, C}, unless differing meaning is explicitly stated or clear from context.

1. A cryotherapeutic system, comprising:
an elongated shaft having a proximal end portion and a distal end portion configured to be delivered to a target site within a bronchial tree beyond the left or right main bronchus, wherein the shaft comprises a delivery line and an exhaust shaft;
a handle disposed at a proximal end of the elongated shaft; and
a cooling assembly at the distal end portion of the elongated shaft, the cooling assembly comprising—
an expandable member having a flexible wall that defines a chamber, wherein the chamber is fluidically connected to the exhaust shaft, and wherein the expandable member has an expanded state in which the expandable member has an outer diameter of at least configured to contact an inner wall of an airway at the target site,
an injection tube extending from the elongated shaft into the chamber, fluidically connected to the delivery line,
wherein the injection tube comprises a plurality of openings configured to dispel refrigerant into the chamber such that the refrigerant expands to impart cryogenic thermal injury to neural fibers positioned a distance away from the inner wall of the airway.

2. The cryotherapeutic system of claim 1 wherein the expandable member comprises a balloon made of a semi-compliant material.

3-5. (canceled)

6. The cryotherapeutic system of claim 1 wherein at least one of the plurality of openings is wedge-shaped and tapers from an inner surface of the injection tube to an outer surface of the injection tube such that a dimension of the opening at the inner surface is larger than a dimension of the opening at the outer surface.

7. The cryotherapeutic system of claim 1 wherein the injection tube comprises a first portion having a first inner diameter and a second portion having a second inner diameter smaller than the first inner diameter, and wherein the plurality of openings are located at or near the second portion.

8-11. (canceled)

12. The cryotherapeutic system of claim 1 wherein the cooling assembly further comprises one or more markers positioned along the injection tube and/or the distal end portion of the elongated shaft.

13. The cryotherapeutic system of claim 1 wherein the cooling assembly further comprises at least one sensor configured to detect at least one of temperature within the expandable member, pressure within the expandable member, and/or flow rate of the refrigerant.

14. The cryotherapeutic system of claim 1, further comprising a protector sheath to slidably extend over at least a portion of the cooling assembly, wherein the protector sheath has a flared proximal region defining an opening larger in cross-sectional dimension than a distal region of the protector sheath.

15. (canceled)

16. The cryotherapeutic system of claim 14, wherein the protector sheath has a distal end region has an outer cross-sectional dimension of at least 2 mm such that at least a portion of the protector sheath is positioned outside a working channel of a bronchoscope when the cooling assembly is positioned within the working channel.

17. (canceled)

18. (canceled)

19. The cryotherapeutic system of claim 1 wherein the cryogenic thermal injury is imparted to neural fibers positioned between 1 mm and 3 mm from the inner wall of the airway.

20. The cryotherapeutic system of claim 1 wherein the handle comprises attachment mechanisms configured to connect the handle to a handle of a bronchoscope.

21. The cryotherapy system of claim 1 wherein the injection tube further comprises a sheath partially surrounding an inner tube, wherein the plurality of openings are located along a length of the inner tube, and wherein the sheath is configured to move with respect to the inner tube to selectively place one or more of the plurality of openings in fluid communication with the chamber.

22. The cryotherapy system of claim 1 wherein the injection tube has a tube distal end with an outlet and a collar extending over the outlet, wherein the plurality of openings are located on the collar, and wherein the collar is movable with respect to the injection tube.

23. The cryotherapy system of claim 1 wherein the cooling assembly further comprises a rotary device operably coupled to the injection tube, wherein the rotary device is configured to rotate a location of the plurality of openings with respect to the expandable member.

24. The cryotherapy system of claim 23 wherein the rotary device comprises a rotary turbine manifold configured to be driven by refrigerant moving through the rotary device.

25. The cryotherapy system of claim 1 wherein the cooling assembly further comprises an ancillary lumen extending at least from a proximal end portion of the expandable member to the distal end portion of the expandable member, and wherein the ancillary lumen is not fluidically coupled to the delivery line.

26. The cryotherapy system of claim 25 wherein the ancillary lumen defines a bypass tube with a distal outlet positioned outside of the chamber and open to an external environment.

27. (canceled)

28. (canceled)

29. The cryotherapy system of claim 1 wherein the injection tube comprises a plurality of individual injection tubes disposed about the exhaust shaft.

30-34. (canceled)

35. The cryotherapy system of claim 1 wherein the injection tube comprises at least one spring section configured to position the plurality of openings along a longitudinal axis of the cooling assembly that is at least partially aligned with an ablation region of the expandable member.

36. The cryotherapy system of claim 1 wherein the injection tube has a curved section configured to stretch or retract to position the plurality of openings in a central portion of the expandable member.

37. (canceled)

38. The cryotherapy system of claim 1 wherein the exhaust shaft comprises a plurality of support wires configured to provide patency of the exhaust shaft.

39. The cryotherapy system of claim 1, further comprising at least one insulating element along the elongated shaft and/or the handle, and wherein the insulating element is configured to maintain a temperature within the exhaust shaft that avoids unregulated expansion of refrigerant flowing through the exhaust shaft.

40. The cryotherapy system of claim 1, further comprising at least one heating element along the elongated shaft and/or the handle, and wherein the heating element is configured to maintain a temperature within the exhaust shaft that avoids unregulated expansion of refrigerant flowing through the exhaust shaft.

41. The cryotherapy system of claim 1 wherein, when the expandable member is deployed, the expandable member is configured to press against and conform to the inner wall of the airway, and wherein the expandable member is configured to treat airways having an inner diameter ranging from at least 3 mm and 10 mm.

42. (canceled)

43. A method of performing targeted lung neuromodulation, the method comprising:
advancing a distal end portion of a cryotherapeutic device through a working channel of a bronchoscope to a target site beyond a left or right main bronchus;
deploying an expandable member of a cooling assembly at the distal end portion at the target site such that the expandable member contacts and confirms to tissue along an inner wall of an airway;
delivering refrigerant through an injection tube into the expandable member, wherein the refrigerant expands within the expandable member to provide cryotherapeutic cooling; and
imparting cryogenic thermal injury to neural fibers positioned a distance away from the inner wall of the airway via the cryotherapeutic cooling.

44. The method of claim 43 wherein delivering the distal end portion to the target site comprises delivery the distal end portion to at least one of a right upper lobe bronchus, a right intermediate bronchus, a left upper lobe bronchus, and/or a left lower lobar bronchus.

45. The method of claim 43, further comprising inflating the expandable member prior to delivering the refrigerant to confirm apposition of the expandable member against the inner wall of the airway.

46. The method of claim 45, further comprising deflating the expandable member and repositioning the expandable member if adequate apposition is not achieved.

47. The method of claim 43, wherein delivering the refrigerant causes ice to form around an exterior of the expandable member, thereby adhering the expandable member to surrounding tissue at the target site.

48. The method of claim 43 wherein imparting cryogenic thermal injury comprises imparting cryogenic thermal injury to the neural fibers positioned between 1 mm and 3 mm from the inner wall of the airway.

49. The method of claim 43, further comprising directly visualizing the target site via the bronchoscope before, during, or after delivering the refrigerant.

50. (canceled)

51. The method of claim 43, further comprising performing multiple ablation cycles of delivering refrigerant that imparts cryogenic thermal injury at the target site by deflating and reinflating the expandable member between cycles.

52. The method of claim 43, further comprising heating a portion of an exhaust shaft as refrigerant moves through the exhaust shaft.

53. The method of claim 43, further comprising applying a cryoprotectant to a portion of the inner wall of the airway before delivering the refrigerant.

54. (canceled)
